(19) United States
(12) Patent Application Publication
(10) Pub. No.: US 2004/0192910 A1
(43) Pub. Date: Sep. 30, 2004

(54) Sulfonylamino derivatives useful as herbicides

(76) Inventors: Christoph Luthy, Basel (CH); Andrew Edmunds, Basel (CH)

Correspondence Address: SYNGENTA CROP PROTECTION , INC., PATENT AND TRADEMARK DEPARTMENT, 410 SWING ROAD, GREENSBORO, NC, 27409, US

(21) Appl. No.: 10/488,262

(22) PCT Filed: Aug. 27, 2002
(86) PCT No.: PCT/EP02/09563

(30) Foreign Application Priority Data
Aug. 28, 2001 (CH) 2001 1591/01

(51) Int. Cl. C07D 279/04; C07D 265/04; C07D 239/02

(52) U.S. Cl. 544/53; 544/88; 544/320

(57) ABSTRACT

Compounds of Formula I (I) wherein the substituents are as defined in claim 1, and the agrochemically tolerable salts and all stereoisomers and tautomers of the compounds of formula I, are suitable for use as herbicides.

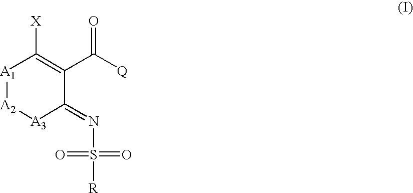

The present invention relates to novel herbicidally active sulfonamino derivatives, to processes for their preparation, to compositions comprising those compounds, and to their use in controlling weeds, especially in crops of useful plants, or in inhibiting plant growth.

Sulfonamino derivatives having herbicidal action are described, for example, in WO 00/15615 and WO 00/39094.

Novel sulfonamino derivatives having herbicidal and growth-inhibiting properties have now been found.

The present invention accordingly relates to compounds of formula I

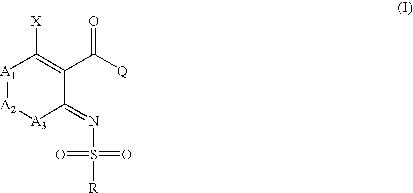

wherein

X is chlorine, OSO₂R or S(O)ₙR₁;

Q is a phenyl group mono- to tetra-substituted by R₂, wherein a saturated or unsaturated 5- to 8-membered ring system may be fused to the phenyl group, which ring system may itself be mono- to tri-substituted by R₁₁ and may contain in the ring one, two or three hetero atom groups selected from —O—, —NR₁₀—, —S(O)ₚ— and —C(X₂)—;

or Q represents a pyridyl or pyridyl-N-oxido group mono- to tri-substituted by R₂, a pyrimidinyl group, or a 5-membered heteroaryl group mono- to tri-substituted by R₂;

A₁ is C(R₃R₄) or NR₂₇:

A₂ is C(R₅R₆)ₘ, C(O), oxygen, NR₇ or S(O)_{q};

A₃ is C(R₈R₉) or NR₂₈;

with the proviso that A₂ is other than NR₇ and S(O)_{q} when A₁ is NR₂₇ and/or A₃ is NR₂₈;

R is C₁-C₁₂alkyl, C₁-C₁₂haloalkyl, C₂-C₁₂alkenyl, C₂-C₁₂haloalkenyl, or is vinyl substituted by C₁-C₂alkoxycarbonyl or by phenyl, or is C₂-C₁₂alkynyl, C₂-C₁₂haloalkynyl, C₃-C₁₂allenyl, C₃-C₆cycloalkyl, NR₁₃R₁₄ or phenyl, wherein the phenyl-containing groups may themselves be mono- to penta-substituted by C₁-C₄alkyl, C₂-C₄alkenyl, C₁-C₄haloalkyl, C₁-C₄alkoxy, C₁-C₄haloalkoxy, halogen, S(O)ᵣR₁₅, S(O)₂NR₁₆R₁₇, cyano, C₁-C₄alkoxycarbonyl, C₁-C₄-alkylcarbonyl, cyclopropylcarbonyl or by nitro;

or R is C₃-C₆cycloalkyl-C₁-C₆alkyl, phenyl-C₁-C₆alkyl, hydroxy-C₁-C₁₂alkyl, C₁-C₄alkoxy-C₁-C₁₂alkyl, C₁-C₄alkylthio-C₁-C₁₂alkyl, C₁-C₄alkylsulfinyl-C₁-C₁₂alkyl, C₁-C₄alkylsulfonyl-C₁-C₁₂alkyl, cyano-C₁-C₁₂alkyl, C₁-C₆alkylcarbonyloxy-C₁-C₁₂alkyl, C₁-C₄alkoxycarbonyl-C₁-C₁₂alkyl, C₁-C₄alkoxycarbonyloxy-C₁-C₁₂alkyl, rhodano-C₁-C₁₂alkyl, benzoyloxy-C₁-C₁₂-alkyl, C₁-C₄alkylamino-C₁-C₁₂alkyl, di-(C₁-C₄alkyl)amino-C₁-C₁₂alkyl, C₁-C₁₂alkylthiocarbonyl-C₁-C₁₂alkyl or formyl-C₁-C₁₂alkyl;

or R is a five- to ten-membered monocyclic or annellated bicyclic ring system, which may be aromatic, partially saturated or fully saturated and may contain from 1 to 4 hetero atoms selected from nitrogen, oxygen and sulfur, the ring system being bonded to the sulfur atom of the S(O)₂N=group either directly or by way of a C₁-C₁₂alkylene chain, and wherein each ring system contains no more than two oxygen atoms and no more than two sulfur atoms, and each ring system may itself be mono- or poly-substituted by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆halo-alkoxy, C₃-C₆alkenyloxy, C₃-C₆alkynyloxy, mercapto, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅-acetylalkylthio, C₃-C₆alkoxycarbonylalkylthio, C₂-C₄cyanoalkylthio, C₁-C₆alkylsulfinyl, C₁-C₆-haloalkylsulfinyl, C₁-C₆alkylsulfonyl, C₁-C₆haloalkylsulfonyl, aminosulfonyl, C₁-C₂alkyl-aminosulfonyl, di-(C₁-C₂alkyl)aminosulfonyl, di-(C₁-C₄alkyl)amino, halogen, cyano, nitro, phenylthio or by benzylthio, wherein the phenylthio and benzylthio groups may themselves be substituted on the phenyl ring by C₁-C₃alkyl, C₁-C₃haloalkyl, C₁-C₃alkoxy, C₁-C₃halo-alkoxy, halogen, cyano or by nitro, and wherein the substituents on the nitrogen in the heterocyclic ringsystem cannot be halogen;

n is 0, 1 or 2;

m is 1 or 2;

p is 0, 1 or 2;

q is 0, 1 or 2;

r is 0, 1 or 2;

R₁ is C₁-C₁₂alkyl, C₁-C₁₂haloalkyl, C₃-C₁₂alkenyl, C₃-C₁₂haloalkenyl, C₃-C₁₂alkynyl, C₃-C₁₂-haloalkynyl, C₃-C₁₂allenyl, C₃-C₆cycloalkyl or phenyl, wherein the phenyl group may itself be mono- to penta-substituted by C₁-C₄alkyl, C₁-C₄haloalkyl, C₁-C₄alkoxy, C₁-C₄haloalkoxy, halogen, S(O)ᵣR₄₁, S(O)₂NR₃₉R₄₀, cyano, C₁-C₄alkoxycarbonyl or by nitro;

or R₁ is phenyl-C₁-C₆alkyl, hydroxy-C₁-C₆alkyl, C₁-C₄alkoxy-C₁-C₈alkyl, C₁-C₄alkylthio-C₁-C₈-alkyl, C₃-C₆alkenylthio-C₃-C₆alkyl, cyano-C₁-C₈alkyl, C₁-C₄alkoxycarbonyl-C₁-C₈alkyl or di-(C₁-C₄alkyl)amino-C₁-C₈alkyl;

each R₂ independently is

hydrogen, halogen, C₁-C₆alkyl, C₂-C₆alkenyl, C₂-C₆haloalkenyl, C₂-C₆alkynyl, C₂-C₆halo-alkynyl, C₃-C₆cycloalkyl, C₁-C₆alkoxy, C₁-C₆haloalkoxy, C₃-C₆alkenyloxy, C₃-C₆alkynyloxy, C₁-C₆alkylthio, C₁-C₆alkylsulfinyl, C₁-C₆alkylsulfonyl, C₁-C₆haloalkyl, C₁-C₆haloalkylthio, C₁-C₆haloalkylsulfinyl, C₁-C₆haloalkylsulfonyl, C₁-C₆alkoxycarbonyl, C₁-C₆alkylcarbonyl, C₁-C₆alkylamino, di-(C₁-C₆alkyl)amino, C₁-C₆alkylaminosulfonyl, di-(C₁-C₆alkyl)aminosulfonyl, —N(R₁₈)—SO₂—R₁₉, nitro, cyano, amino, formyl, hydroxy-C₁-C₆alkyl, C₁-C₆alkoxy-C₁-C₆alkyl, C₁-C₆haloalkoxy-C₁-C₆alkyl, C₁-C₈alkylcarbonyloxy-C₁-C₄alkyl, C₁-C₆alkoxycarbonyloxy-C₁-C₆alkyl, C₁-C₆alkylthio-C₁-C₆alkyl, C₁-C₆alkylsulfinyl-C₁-C₆alkyl, C₁-C₆alkylsulfonyl-C₁-C₆-alkyl, rhodano-C₁-C₆alkyl, cyano-C₁-C₆alkyl, C₃-C₆alkenyloxy-C₁-C₃alkyl, C₃-C₆alkynyloxy-C₁-C₃alkyl, C₁-C₆alkoxy-C₁-C₆alkoxy-C₁-C₃alkyl, C₁-C₆haloalkoxy-C₁-C₆alkoxy-C₁-C₆alkyl, C₁-C₄alkoxy-C₁-C₄alkoxy-C₁-C₄alkoxy-C₁-C₃alkyl, C₁-C₄alkoxy-C₁-C₄alkoxy-C₁-C₄alkoxy-C₁-C₄alkoxy-C₁-C₃alkyl, C₁-C₈alkylcarbonyloxy-C₁-C₄alkoxy-C₁-C₄alkyl, C₁-C₆alkoxy-C₁-C₆-alkoxy, C₁-C₆alkoxycarbonyloxy-C₁-C₆alkoxy, cyano-C₁-C₆alkoxy, cyano-C₁-C₆alkenyloxy, C₁-C₆alkoxycarbonyl-C₁-C₆alkoxy-C₁-C₃alkyl, C₁-C₆alkoxycarbonyl-C₁-C₆alkoxy, C₁-C₆-alkylthio-C₁-C₆alkoxy, C₁-C₆alkylthio-C₁-C₆alkoxy-C₁-C₃alkyl, C₁-C₆alkoxycarbonyl-C₁-C₆-alkylthio, C₁-C₆alkoxycarbonyl-C₁-C₆alkylthio-C₁-C₃alkyl, C₁-C₆alkoxycarbonyl-C₁-C₆-alkylsulfinyl, C₁-C₆alkoxycarbonyl-C₁-C₆alkylsulfinyl-C₁-C₃alkyl, C₁-C₆alkoxycarbonyl-C₁-C₆-alkylsulfonyl, C₁-C₆alkoxycarbonyl-C₁-C₆alkylsulfonyl-C₁-C₃alkyl, N—(C₁-C₆alkyl)-C₁-C₆-alkylsulfonylamino-C₁-C₃alkyl, C₁-C₆alkylsulfonyloxy, C₁-C₆haloalkylsulfonyloxy, phenyl, benzyl, phenoxy, phenylthio, phenylsulfinyl, phenylsulfonyl, benzylthio, benzylsulfinyl or benzylsulfonyl, wherein the phenyl-containing groups may themselves be mono-, di- or tri-substituted by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, C₁-C₆-alkylthio, C₁-C₆haloalkylthio, C₃-C₆alkenylthio, C₃-C₆haloalkenylthio, C₃-C₆alkynylthio, C₁-C₄-alkoxy-C₁-C₃alkylthio, C₁-C₄alkylcarbonyl-C₁-C₃alkylthio, C₁-C₄alkoxycarbonyl-C₁-C₃alkylthio, C₂-C₄cyano-C₁-C₃alkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di-(C₁-C₄alkyl)aminosulfonyl, C₁-C₃alkoxy-C₁-C₃alkyl, C₁-C₃alkoxycarbonyl-C₁-C₃alkyl, C₁-C₃alkylthio-C₁-C₃alkyl, alkyl-sulfinyl-C₁-C₃alkyl, alkylsulfonyl-C₁-C₃alkyl, NR₃₀R₃₁, halogen, cyano, nitro, phenyl, or by phenyl-C₁-C₃alkylene which may be interrupted by oxygen or by —S(O)ₚ—, wherein the phenyl and phenyl-C₁-C₃alkylene groups may be substituted on the phenyl ring by C₁-C₃alkyl, C₁-C₃haloalkyl, C₁-C₃alkoxy, C₁-C₃haloalkoxy, halogen, cyano or by nitro;

or each R₂ independently is C₃-C₆cycloalkyl which is bonded to the group Q either directly or by way of a C₁-C₄alkylene chain which may be interrupted by oxygen or by S(O)ₚ, wherein cycloalkyl may be substituted by C₁-C₃alkyl or by halogen, or

each R₂ independently is phenyl which is bonded to the group 0 by way of a C₁-C₄alkylene chain (which may additionally be interrupted by oxygen or by S(O)p) and which may be substituted by C₁-C₃alkyl, C₁-C₃haloalkyl, C₁-C₃alkoxy, C₁-C₃haloalkoxy, halogen, cyano or by nitro;

or each R₂ independently is a five- to ten-membered monocyclic or annellated bicyclic ring system, which may be aromatic, partially saturated or fully saturated and may contain from 1 to 4 hetero atoms selected from nitrogen, oxygen and sulfur or a carbonyl group; the ring system being bonded to the group Q either directly or by way of a C₁-C₄alkylene chain which may be interrupted by oxygen or by —S(O)ₚ—, and wherein each ring system may contain no more than two oxygen atoms and no more than two sulfur atoms, and the ring system may itself be mono-, di- or tri-substituted by halogen, C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, C₁-C₆alkylthio, C₁-C₆haloalkylthio, C₃-C₆alkenylthio, C₃-C₆haloalkenylthio, C₃-C₆alkynylthio, C₂-C₅alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₄alkylaminosulfonyl, C₁-C₄di-alkylaminosulfonyl, R₂₀-C₁-C₃alkylene, NR₃₂R₃₃, cyano, nitro, phenylthio or by benzylthio, wherein phenylthio and benzylthio may themselves be substituted on the phenyl ring by C₁-C₃alkyl, C₁-C₃haloalkyl, C₁-C₃alkoxy, C₁-C₃haloalkoxy, halogen, cyano or by nitro, and wherein the substituents on the nitrogen in the heterocyclic ringsystem cannot be halogen;

R₃ is hydrogen, C₁-C₆alkyl, C₂-C₆alkenyl, C₂-C₆alkynyl, C₁-C₆alkoxy, C₁-C₆alkyl-S(O)ᵣ—, C₁-C₆alkoxycarbonyl, C₁-C₆alkylcarbonyl, C₁-C₆alkyl-NHS(O)₂, C₁-C₆alkylamino, di-(C₁-C₆-alkyl)amino, hydroxy, C₁-C₆alkoxy, C₃-C₆alkenyloxy, C₃-C₆alkynyloxy, hydroxy-C₁-C₆alkyl, C₁-C₄alkylsulfonyloxy-C₁-C₆alkyl, tosyloxy-C₁-C₆alkyl, halogen, cyano, nitro, phenyl or phenyl substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, amino, C₁-C₄alkylamino, di-C₁-C₄alkylamino, C₁-C₄alkylthio, C₁-C₄-alkylsulfinyl, C₁-C₄alkylsulfonyl, C₁-C₄alkylsulfonyloxy, C₁-C₄haloalkylthio, C₁-C₄haloalkyl-sulfinyl, C₁-C₄haloalkylsulfonyl, C₁-C₄haloalkylsulfonyloxy, C₁-C₄alkylsulfonylamino, N—(C₁-C₄alkyl)-C₁-C₄alkylsulfonylamino, halogen, nitro, COOH or cyano;

R₄ is hydrogen, C₁-C₄alkyl or C₁-C₄alkyl-S(O)ᵣ—;

or R₄ together with R₃ is a C₂-C₅alkylene chain which may be interrupted by —O—, —C(O)—, —O—, —C(O)— or —S(O)ₚ—;

R₅ is hydrogen, hydroxy, C₁-C₆alkyl, C₁-C₆haloalkyl, C₁-C₄hydroxyalkyl, C₁-C₄alkoxy-C₁-C₄-alkyl, C₁-C₄alkylthio-C₁-C₄alkyl, C₁-C₄alkylthio-C₃-C₆cycloalkyl, C₁-C₄alkylcarbonyloxy-C₁-C₄-alkyl, C₁-C₄alkylsulfonyloxy-C₁-C₄alkyl, tosyloxy-C₁-C₄alkyl, di-(C₁-C₄alkoxy)-C₁-C₄alkyl, C₁-C₄alkoxycarbonyl, di-(C₁-C₃alkylthio)-C₁-C₄alkyl, (C₁-C₃alkoxy)-(C₁-C₃alkylthio)-C₁-C₄alkyl, C₃-C₅oxacycloalkyl, C₃-C₅thiacycloalkyl, C₃-C₄dioxacycloalkyl, C₃-C₄dithiacycloalkyl, C₃-C₄oxathiacycloalkyl, formyl, C₁-C₄alkoxyiminomethyl, carbamoyl, C₁-C₄alkylamino-carbonyl, di-(C₁-C₄alkyl)aminocarbonyl, phenylaminocarbonyl, benzylaminocarbonyl or phenyl, wherein the phenyl-containing groups may themselves be substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, amino, C₁-C₄alkylamino, di-C₁-C₄alkylamino, C₁-C₄alkylthio, C₁-C₄alkylsulfinyl, C₁-C₄alkyl-sulfonyl, C₁-C₄alkylsulfonyloxy, C₁-C₄haloalkylthio, C₁-C₄haloalkylsulfinyl, C₁-C₄haloalkyl-sulfonyl, C₁-C₄haloalkylsulfonyloxy, C₁-C₄alkylsulfonylamino, N—(C₁-C₄alkyl)-C₁-C₄alkyl-sulfonylamino, halogen, nitro, COOH or by cyano;

or R₅ together with R₃, R₄, R₈, R₉, R₂₇ or R₂₈ may form a direct bond or a C₁-C₄alkylene bridge, or, when m is 2, two substituents R₅ together may form a direct bond;

R₆ is hydrogen, C₁-C₄alkyl or C₁-C₄haloalkyl;

R₇ is hydrogen, C₁-C₄alkyl, C₁-C₄alkoxycarbonyl, C₁-C₄alkylcarbonyl or di-(C₁-C₄alkyl)amino-carbonyl, or phenyl which may itself be substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, C₁-C₄alkylamino, di-C₁-C₄alkylamino, C₁-C₄alkylthio, C₁-C₄alkylsulfinyl, C₁-C₄alkylsulfonyl, C₁-C₄alkylsulfonyloxy, C₁-C₄haloalkylthio, C₁-C₄haloalkylsulfinyl, C₁-C₄haloalkylsulfonyl, C₁-C₄haloalkylsulfonyloxy, C₁-C₄alkylsulfonylamino, N—(C₁-C₄alkyl)-C₁-C₄alkylsulfonylamino, halogen, nitro or by cyano;

R₈ is hydrogen, C₁-C₆alkyl, C₂-C₆alkenyl, C₂-C₆alkynyl, C₁-C₆alkoxy, C₁-C₆alkyl-S(O)ᵣ, C₁-C₆-alkoxycarbonyl, C₁-C₆alkylcarbonyl, C₁-C₆alkylaminosulfonyl, C₁-C₆alkylamino, di-(C₁-C₆-alkyl)amino, hydroxy, C₁-C₆alkoxy, C₃-C₆alkenyloxy, C₃-C₆alkynyloxy, hydroxy-C₁-C₆alkyl, C₁-C₄alkylsulfonyloxy-C₁-C₆alkyl, tosyloxy-C₁-C₆alkyl, halogen, cyano, nitro, phenyl, or phenyl substituted by C₁-C₄alkyl, C₁-C₄haloalkyl, C₁-C₄alkoxy, C₁-C₄haloalkoxy, C₁-C₄alkyl-carbonyl, C₁-C₄alkoxycarbonyl, amino, C₁-C₄alkylamino, di-(C₁-C₄alkyl)amino, C₁-C₆alkylthio, C₁-C₆alkylsulfinyl, C₁-C₆alkylsulfonyl, C₁-C₄alkylsulfonyloxy, C₁-C₆haloalkylthio, C₁-C₆halo-alkylsulfinyl, C₁-C₆haloalkylsulfonyl, C₁-C₄haloalkylsulfonyloxy, C₁-C₄alkylsulfonylamino, N—(C₁-C₄alkyl)-C₁-C₄alkylsulfonylamino, halogen, nitro, COOH or by cyano;

or, when A₂ is C(R₅R₆)1, R₈ together with R₃ or R₄ may form a direct bond or a C₁-C₃alkylene bridge;

R₉ is hydrogen or C₁-C₄alkyl;

or R₉ together with R₈ forms a C₂-C₅alkylene bridge which may be interrupted by —O—, —C(O)—, —O—, —C(O)— or —S(O)ₚ—;

R₁₀ is hydrogen or C₁-C₆alkyl;

R₁₁ is halogen, C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, C₁-C₆-alkylthio, C₁-C₆haloalkylthio, C₃-C₆alkenylthio, C₃-C₆haloalkenylthio, C₃-C₆alkynylthio, C₁-C₄-alkoxy-C₁-C₂alkylthio, C₁-C₄alkylcarbonyl-C₁-C₂alkylthio, C₁-C₄alkoxycarbonyl-C₁-C₂alkylthio, cyano-C₁-C₄alkylthio, C₁-C₆alkylsulfinyl, C₁-C₆haloalkylsulfinyl, C₁-C₆alkylsulfonyl, C₁-C₆-haloalkylsulfonyl, aminosulfonyl, C₁-C₄alkylaminosulfonyl, di-(C₁-C₄alkyl)aminosulfonyl, R₂₉-C₁-C₃alkylene, NR₃₄R₃₅, halogen, cyano, nitro, phenylthio or benzylthio, wherein phenylthio and benzylthio may themselves be mono-, di- or tri-substituted on the phenyl ring by C₁-C₃alkyl, C₁-C₃haloalkyl, C₁-C₃alkoxy, C₁-C₃haloalkoxy, halogen, cyano or by nitro;

X₂ is oxygen, di-C₁-C₄alkoxy or ═NR₁₂;

R₁₂ is hydroxy or C₁-C₄alkoxy; R₁₃, R₁₄, R₁₅, R₁₆, R₁₇, R₃₀, R₃₁, R₃₂, R₃₃, R₃₄, R₃₅, R₃₆, R₃₇, R₃₈, R₃₉, R₄₀ and R₄, are each independently of the others C₁-C₁₂alkyl;

or R₁₃ and R₁₄ together or R₃₀ and R₃₁ together or R₃₂ and R₃₃ together or R₃₄ and R₃₅ together or R₃₆ and R₃₇ together and/or R₁₆ and R₁₇ together or R₃₉ and R₄₀ together form, with the nitrogen atom to which they are bonded, a 3- to 7-membered ring;

R₁₈ is hydrogen or C₁-C₆alkyl;

R₁₉ is NR₃₇R₃₈, C₁-C₆alkyl, C₁-C₆haloalkyl, C₂-C₆alkenyl, C₂-C₆haloalkenyl, C₃-C₆alkynyl, C₃-C₆haloalkynyl, C₃-C₆cycloalkyl or phenyl, wherein phenyl may itself be substituted by C₁-C₃alkyl, C₁-C₃haloalkyl, C₁-C₃alkoxy, C₁-C₃haloalkoxy, halogen, cyano or by nitro;

R₂₀ and R₂₉ are each independently of the other C₁-C₃alkoxy, C₂-C₄alkoxycarbonyl, C₁-C₃-alkylthio, C₁-C₃alkylsulfinyl, C₁-C₃alkylsulfonyl or phenyl, wherein phenyl may itself be substituted by C₁-C₃alkyl, C₁-C₃haloalkyl, C₁-C₃alkoxy, C₁-C₃haloalkoxy, halogen, cyano or by nitro;

R₂₇ and R₂₈ are each independently of the other hydrogen, C₁-C₆alkyl, C₁-C₆haloalkyl, C₃-C₆alkenyl, C₃-C₆alkynyl, C₁-C₆alkoxy, benzyl or phenyl, wherein benzyl or phenyl may themselves be substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, C₁-C₄alkylthio, C₁-C₄alkylsulfinyl, C₁-C₄alkyl-sulfonyl, C₁-C₄haloalkylthio, C₁-C₄haloalkylsulfinyl, C₁-C₄haloalkylsulfonyl, halogen, nitro or by cyano;

and to the agrochemically tolerable salts and all stereoisomers and tautomers of the compounds of formula I.

The alkyl groups mentioned in the substituent definitions may be straight-chain or branched and are, for example, methyl, ethyl, n-propyl, isopropyl, n-butyl, sec-butyl, isobutyl, tert-butyl, pentyl, hexyl, heptyl, octyl, nonyl, decyl, undecyl and dodecyl and branched isomers thereof. Alkoxy, alkenyl and alkynyl radicals are derived from the mentioned alkyl radicals. The alkenyl and alkynyl groups may be mono- or poly-unsaturated, with C₂-C₁₂alkyl chains having one or more double or triple bonds also being included. Alkenyl is, for example, vinyl, allyl, isobuten-3-yl, CH₂═CH—CH₂—CH═CH₂—, CH₂═CH—CH₂—CH₂—CH═CH₂— or CH₃—CH═CH—CH₂—CH═CH—. Alkynyl is, for example, propargyl and allenyl is CH₂═C═CH₂—.

An alkylene chain can be substituted by one or more C₁-C₃alkyl groups, especially methyl groups; such alkylene chains and alkylene groups are preferably unsubstituted. The same also applies to all groups containing C₃-C₆cycloalkyl, C₃-C₅oxacycloalkyl, C₃-C₅thiacycloalkyl, C₃-C₄dioxacycloalkyl, C₃-C₄dithiacycloalkyl or C₃-C₄oxathiacycloalkyl.

A C₁-C₄alkylene chain which may be interrupted by oxygen or by S(O)p is to be understood as being, for example, H₂—, —CH₂O—, —OCH₂—, —CH₂OCH₂—, —OCH₂CH₂— or —CH₂SCH₂—.

Halogen is generally fluorine, chlorine, bromine or iodine, preferably fluorine or chlorine. The same is also true of halogen in connection with other definitions such as haloalkyl, halo-alkoxy or halophenyl.

Haloalkyl groups having a chain length of from 1 to 6 carbon atoms are, for example fluoro-methyl, difluoromethyl, trifluoromethyl, chloromethyl, dichloromethyl, trichloromethyl, 2,2,2-trifluoroethyl, 1-fluoroethyl, 2-fluoroethyl, 2-chloroethyl, 2-fluoroprop-2-yl, pentafluoroethyl, 1,1-difluoro-2,2,2-trichloroethyl, 2,2,3,3-tetrafluoroethyl and 2,2,2-trichloroethyl, pentafluoro-ethyl, heptafluoro-n-propyl, perfluoro-n-hexyl. Preferred haloalkyl groups in the meanings R to Rₓ, and especially for at least one R₂ group, when Q is a pyridyl or pyridyl-N-oxido group, are fluoromethyl, difluoromethyl, difluorochloromethyl, trifluoromethyl and pentafluoroethyl.

As haloalkenyl there come into consideration alkenyl groups mono- or poly-substituted by halogen, where halogen is fluorine, chlorine, bromine or iodine and especially fluorine or chlorine, for example 1-chlorovinyl, 2-chlorovinyl, 2,2-difluoro-vinyl, 2,2-difluoro-prop-1-en-2-yl, 2,2-dichloro-vinyl, 3-fluoroprop-1-enyl, chloroprop-1-en-1-yl, 3-bromoprop-1-en-1-yl, 2,3,3-trifluoroprop-2-en-1-yl, 2,3,3-trichloroprop-2-en-1-yl and 4,4,4-trifluoro-but-2-en-1-yl.

As haloalkynyl there come into consideration, for example, alkynyl groups mono- or poly-substituted by halogen, where halogen is bromine, iodine and especially fluorine or chlorine, for example 3-fluoropropynyl, 3-chloropropynyl, 3-bromopropynyl, 3,3,3-trifluoropropynyl and 4,4,4-trifluoro-but-2-yn-1-yl.

A C₃-C₆cycloalkyl group may likewise be mono- or poly-substituted by halogen, for example 2,2-dichlorocyclopropyl, 2,2-dibromocyclopropyl, 2,2,3,3-tetrafluorocyclobutyl or 2,2-difluoro-3,3-dichlorocyclobutyl.

Alkoxy groups preferably have a chain length of from 1 to 6 carbon atoms. Alkoxy is, for example, methoxy, ethoxy, propoxy, isopropoxy, n-butoxy, isobutoxy, sec-butoxy and tert-butoxy as well as the pentyloxy and hexyloxy isomers; preferably methoxy and ethoxy.

Haloalkoxy groups preferably have a chain length of from 1 to 6 carbon atoms, for example fluoromethoxy, difluoromethoxy, trifluoromethoxy, 2,2,2-trifluoroethoxy, 1,1,2,2-tetrafluoro-ethoxy, 1-fluoroethoxy, 2-fluoroethoxy, 2-chloroethoxy, 2,2-difluoroethoxy and 2,2,2-trichloro-ethoxy; preferably fluoromethoxy, difluoromethoxy, 2-chloroethoxy and trifluoromethoxy.

Alkylthio groups preferably have a chain length of from 1 to 8 carbon atoms. Alkylthio is, for example, methylthio, ethylthio, propylthio, isopropylthio, n-butylthio, isobutylthio, sec-butylthio or tert-butylthio, preferably methylthio or ethylthio. Alkylsulfinyl is, for example, methylsulfinyl, ethylsulfinyl, propylsulfinyl, isopropylsulfinyl, n-butylsulfinyl, isobutylsulfinyl, sec-butylsulfinyl, tert-butylsulfinyl; preferably methylsulfinyl or ethylsulfinyl.

Alkylsulfonyl is, for example, methylsulfonyl, ethylsulfonyl, propylsulfonyl, isopropylsulfonyl, n-butylsulfonyl, isobutylsulfonyl, sec-butylsulfonyl or tert-butylsulfonyl; preferably methyl-sulfonyl or ethylsulfonyl.

Alkylamino is, for example, methylamino, ethylamino, n-propylamino, isopropylamino or the butylamine isomers. Dialkylamino is, for example, dimethylamino, methylethylamino, diethylamino, n-propylmethylamino, dibutylamino or diisopropylamino. Alkylamino groups having a chain length of from 1 to 4 carbon atoms are preferred.

Alkoxyalkyl groups have preferably from 2 to 6 carbon atoms. Alkoxyalkyl is, for example, methoxymethyl, methoxyethyl, ethoxymethyl, ethoxyethyl, n-propoxymethyl, n-propoxyethyl, isopropoxymethyl or isopropoxyethyl.

Alkoxyalkoxyalkyl groups have preferably from 3 to 8 carbon atoms, for example methoxy-methoxymethyl, methoxyethoxymethyl, ethoxymethoxymethyl, ethoxyethoxymethyl.

Alkylthioalkyl groups have preferably from 2 to 6 carbon atoms. Alkylthioalkyl is, for example, methylthiomethyl, methylthioethyl, ethylthiomethyl, ethylthioethyl, n-propylthiomethyl, n-propylthioethyl, isopropylthiomethyl, isopropylthioethyl, butylthiomethyl, butylthioethyl or butylthiobutyl.

Alkylcarbonyl is preferably acetyl or propionyl.

Alkoxycarbonyl is, for example, methoxycarbonyl, ethoxycarbonyl, propoxycarbonyl, iso-propoxycarbonyl, n-butoxycarbonyl, isobutoxycarbonyl, sec-butoxycarbonyl or tert-butoxy-carbonyl; preferably methoxycarbonyl, ethoxycarbonyl or tert-butoxycarbonyl.

Phenyl, also as part of a substituent such as phenoxy, benzyl, benzyloxy, benzoyl, phenyl-thio, phenylalkyl, phenoxyalkyl or tosyl, may be mono- or poly-substituted. The substituents may then be in the ortho-, meta- and/or para-position, as desired.

Q as a pyridyl group may be a 2-pyridyl, 3-pyridyl or 4-pyridyl group, with special mention being made of the 3-pyridyl group and the 3-pyridyl N-oxide group. Q as a pyrimidinyl group may be a 2-, 4- or 5-pyrimidinyl group, with special mention being made of the 5-pyrimidinyl group.

Q as a substituted 5-membered heteroaryl group may be, for example, furyl, thienyl, pyrrolyl, pyrazolyl, imidazolyl, triazolyl, isoxazolyl, oxazolyl or thiazolyl.

A five- to ten-membered monocyclic or annellated bicyclic ring system, which may be aromatic, partially saturated or fully saturated and may contain from 1 to 4 hetero atoms selected from nitrogen, oxygen and sulfur or a carbonyl group, and wherein each ring system may contain no more than two oxygen atoms and no more than two sulfur atoms, is to be understood as being, for example, pyridyl, pyrimidinyl, triazinyl, pyrrolyl, pyrazolyl, triazolyl, tetrazolyl, thienyl, furyl, isoxazolyl, oxadiazolyl, thiazolyl, thiadiazolyl, 2-pyranyl, 1,3-dioxol-2-yl, oxiranyl, 3-oxetanyl, tetrahydrofuranyl, tetrahydropyranyl, or the group

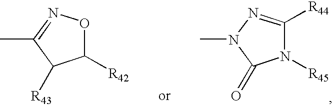

wherein R₄₂ and R₄₃ are each independently of the other preferably hydrogen, halogen, C₁-C₄alkyl, C₁-C₄haloalkyl, C₁-C₄alkoxy or C₁-C₄alkylthio, or together are a C₁-C₄alkylene group; R₄₄ is especially hydrogen, halogen, C₁-C₄alkyl or C₁-C₄alkoxy; and R₄₅ is preferably hydrogen or C₁-C₄alkyl.

A further fused-on, saturated or unsaturated ring system, which is formed by two adjacent substituents R₂ of the phenyl, pyridyl or heteroaryl group Q and which may be interrupted one or more times by —O—, —NR₁₂—, —S(O)ₚ— or by —C(X₂)— and which may be substituted by one or more substituents R₁₁, is to be understood as being, for example, an annellated, bidentate ring system of the form

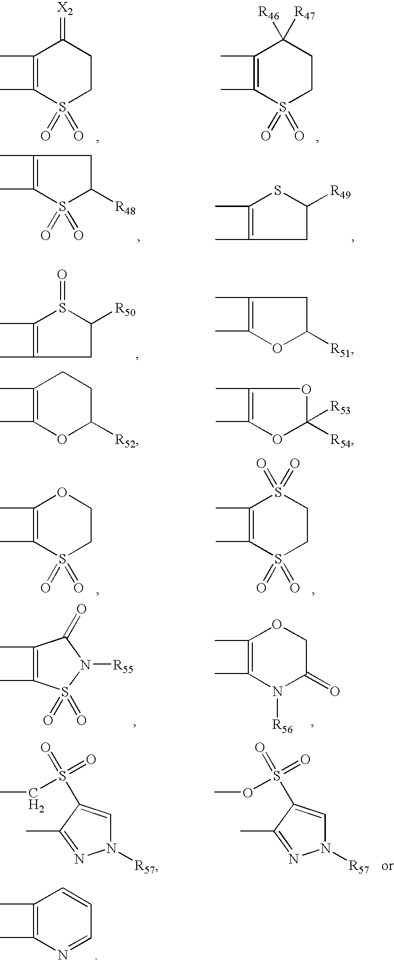

In those formulae, the substituents R₄₆ to R58 represent preferred meanings and positions of the substituents R₁₁.

R₄₆ is hydrogen, halogen, C₁-C₄alkyl, C₁-C₄haloalkyl, C₁-C₄alkoxy or C₁-C₄alkylthio;

R₄₇ is especially hydrogen, halogen, C₁-C₄alkyl, C₁-C₄alkoxy; and

R₅₀, R₅₁, R₅₂, R₅₄, R₅₅, R₅₆, R₅₇ and R₅₈ are hydrogen or C₁-C₄alkyl.

The present invention also includes the salts which the compounds of formula I, and especially the compounds of formula Ia, are able to form with amines, alkali metal and alkaline earth metal bases or quaternary ammonium bases. Of the alkali metal and alkaline earth metal bases, special mention as salt-formers is to be made of the hydroxides of lithium, sodium, potassium, magnesium or calcium, especially those of sodium or potassium. Examples of amines suitable for ammonium salt formation are both ammonia and primary, secondary and tertiary C₁-C₁₈alkylamines, C₁-C₄hydroxyalkylamines and C₂-C₄alkoxyalkyl-amines, for example methylamine, ethylamine, n-propylamine, isopropylamine, the four isomers of butylamine, n-amylamine, isoamylamine, hexylamine, heptylamine, octylamine, nonylamine, decylamine, pentadecylamine, hexadecylamine, heptadecylamine, octa-decylamine, methylethylamine, methylisopropylamine, methylhexylamine, methylnonyl-amine, methylpentadecylamine, methyloctadecylamine, ethylbutylamine, ethylheptylamine, ethyloctylamine, hexylheptylamine, hexyloctylamine, dimethylamine, diethylamine, di-n-propylamine, diisopropylamine, di-n-butylamine, di-n-amylamine, diisoamylamine, dihexyl-amine, diheptylamine, dioctylamine, ethanolamine, n-propanolamine, isopropanolamine, N,N-diethanolamine, N-ethylpropanolamine, N-butylethanolamine, allylamine, n-butenyl-2-amine, n-pentenyl-2-amine, 2,3-dimethylbutenyl-2-amine, dibutenyl-2-amine, n-hexenyl-2-amine, propylenediamine, trimethylamine, triethylamine, tri-n-propylamine, triisopropylamine, tri-n-butylamine, triisobutylamine, tri-sec-butylamine, tri-n-amylamine, methoxyethylamine and ethoxyethylamine; heterocyclic amines, for example pyridine, quinoline, isoquinoline, morpholine, piperidine, pyrrolidine, indoline, quinuclidine and azepine; primary arylamines, for example anilines, methoxyanilines, ethoxyanilines, o-, m- and p-toluidines, phenylene-diamines, naphthylamines and o-, m- and p-chloroanilines; but especially triethylamine, iso-propylamine and diisopropylamine. Quaternary ammonium bases suitable for salt formation are, for example, [N(RₐR_{b}R_{c}R_{d}]⁺ OH⁻, wherein Rₐ, R_{b}, R_{c} and R_{d} are each independently of the others C₁-C₄alkyl. Further suitable tetraalkylammonium bases with other anions can be obtained, for example, by anion exchange reactions. M⁺ is preferably an ammonium salt, especially NH₄⁺, or an alkali metal, especially potassium or sodium.

Depending on the preparation process, the compounds of formula I and, especially, the intermediates of formula Ia (compounds of formula I wherein X is hydroxy) can occur in various tautomeric forms, for example in the form of formula A or formula B, with formula A being preferred.

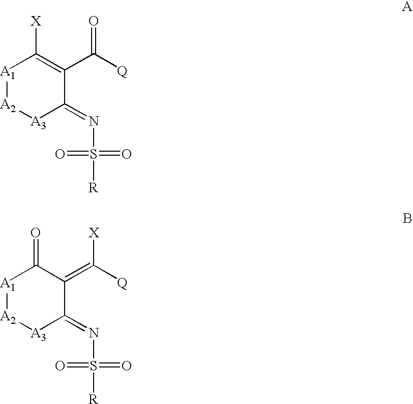

The C═N double bonds in the group C═N—SO₂R in formula I may be either in the E form or in the Z form. The present invention also includes all those stereoisomeric forms of the compound of formula I.

Of the compounds of formula I, special preference is given to those groups wherein:

a) A₁ is CR₃R₄ and R₃ is hydrogen, methyl, ethyl, propargyl, methoxycarbonyl, ethoxy-carbonyl, methylthio, methylsulfinyl or methylsulfonyl and R₄ is hydrogen or methyl, or R₄ together with R₃ is an ethylene bridge;

b) A₂ is CR₅R₆ or an ethylene bridge —(CH₂)₂—, and R₅ is hydrogen, methyl or trifluoromethyl and R₆ is hydrogen or methyl, or R₅ together with R₄ or R₉ is a direct bond or a methylene bridge;

c) A₂ is C(O) and R₃, R₄, R₈ and R₉ are each methyl;

d) A₂ is oxygen and R₃, R₄, R₈ and R₉ are each methyl, or R₈ together with R₄ forms an ethylene bridge and R₃ and RB are hydrogen;

e) A₃ is CR₈R₉ and R₈ and R₉ are hydrogen or methyl, or R₈ together with R₄ is a methylene bridge or an ethylene bridge;

f) R is C₁-C₁₂alkyl, C₁-C₄haloalkyl, preferably trifluoromethyl, C₂-C₁₂alkenyl, C₂-C₁₂halo-alkenyl, benzyl, phenyl, pyridyl, pyrazolyl or thienyl; wherein the phenyl, pyridyl, pyrazolyl or thienyl containing groups may themselves be substituted by C₁-C₄alkyl, C₁-C₄haloalkyl, C₁-C₄alkoxy, C₁-C₄haloalkoxy, halogen, S(O)ᵣR₁₅, S(O)₂NR₁₆R₁₇, C₁-C₄alkoxycarbonyl, cyano or by nitro;

g) Q is phenyl mono-, di-, tri- or tetra-substituted by R₂ and wherein at least one group R₂ is in the ortho-position with respect to the carbonyl group C(O)Q;

h) Q is a di- or tri-substituted phenyl radical and R₂ in the 2-position is preferably methyl, trifluoromethyl, chlorine, bromine, methoxy, methoxymethyl, methylthio, methyl-sulfinyl, methylsulfonyl, cyano or nitro, and in the 4-position is preferably trifluoro-methyl, chlorine, bromine, cyano, nitro, methylsulfonyl, methylsulfonyloxy or methylsulfonylamino, and in the 3-position with respect to the substituent —C(O)-Q may contain a further substituent R₂;

i) Q is a phenyl group, wherein a group R₂ in the 2-position with respect to the substituent —C(O)-Q is substituted by methyl, halomethyl, chlorine or by bromine, and in the 3,4-positions with respect to the substituent —C(O)-Q is substituted by an annellated ring system, such as preferably by the bidentate groups —OCH₂O—, —S(O)ₚCH₂CH₂—, —S(O)ₚCH(CH₃)CH₂—, —CH₂CH₂CH₂S(O)ₚ, —CH(CH₃)CH₂CH₂S(O)ₚ—, —CH(OCH₃)CH₂CH₂S(O)ₚ—, —C(O)CH₂CH₂S(O)ₚ—, —C(OCH₃)₂CH₂CH₂S(O)ₚ—, —C(NOH)CH₂CH₂S(O)ₚ—, —C(NOCH₃)CH₂CH₂S(O)ₚ— or —SO₂N(CH₃)C(O)—, wherein p is 0, 1 or 2.

j) Q is a phenyl group, wherein a group R₂ in the 4-position with respect to the substituent —C(O)-Q is halomethyl, chlorine, bromine, nitro, methylthiomethyl, methyl-sulfinylmethyl, methylsulfonyl, methylsulfonyloxy, methylsulfonylamino, halomethyl-sulfonylamino, and in the 2,3-positions with respect to the substituent —C(O)-Q is substituted by an annellated bidentate ring system such as, preferably, —S(O)ₚCH₂CH₂—, —S(O)ₚCH(CH₃)CH₂—, —SO₂N(CH₃)C(O)—, —CH₂CH₂O—, —CH₂CH(CH₃)O—, —CH₂CH₂CH₂O—, —CH₂CH₂CH(CH₃)O— or —CH₂CH(CH₂OCH₃)O—, wherein p is 0, 1 or 2;

k) 0 is a mono- or di-substituted pyridyl or pyridyl N-oxide group, especially a 3-pyridyl group substituted in the 2,4- or 2,6-positions;

l) Q is a 3-pyridyl group substituted in the 2-position by R₂ as defined in formula I, preferably in the 2-position by R₂ as C₁-C₃alkyl, fluoromethyl, difluoromethyl, trifluoro-methyl, C₁-C₃alkoxy-C₁-C₂alkyl, C₁-C₃alkoxy-C₁-C₃alkoxy-C₁-C₃alkyl, C₁-C₃alkylthio-C₁-C₂alkyl, N—(C₁-C₃alkyl)-C₁-C₃alkylsulfonylaminomethyl, and/or in the 6-position by R₂ as difluoromethyl, difluorochloromethyl, trifluoromethyl or pentafluoroethyl;

m) X is chlorine;

n) X is S(O)ₙR₁, and n is 0 and R₁ is C₁-C₁₂alkyl, C₂-C₁₂alkenyl or phenyl, wherein the phenyl group may itself be substituted by C₁-C₄alkyl, C₁-C₄haloalkyl, C₁-C₄alkoxy, C₁-C₄haloalkoxy, halogen, cyano or by nitro.

An especially preferred group of compounds of formula I is formed by compounds of formula Ib

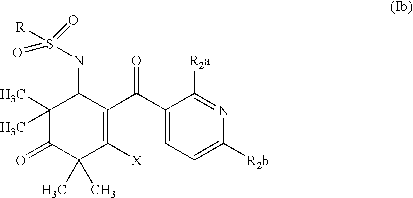

wherein

X is chlorine, C₁-C₈alkylthio, C₃-C₆alkenylthio or phenylthio;

R₂a is C₁-C₃alkyl, fluoromethyl, difluoromethyl, trifluoromethyl, C₁-C₃alkoxymethyl or C₁-C₂-alkoxy-C₁-C₂alkoxymethyl;

R₂b is difluoromethyl, difluorochloromethyl or trifluoromethyl, most especially preferably trifluoromethyl; and

R is C₁-C₆alkyl, C₂-C₆alkenyl, C₁-C₄haloalkyl, phenyl, pyridyl, or phenyl or pyridyl substituted by halogen, C₁-C₄alkyl, C₁-C₄alkoxycarbonyl, C₁-C₄haloalkoxy or by C₁-C₄haloalkyl.

The compounds of formula I can be prepared by processes known per se, for example, for the preparation of a compound of formula I

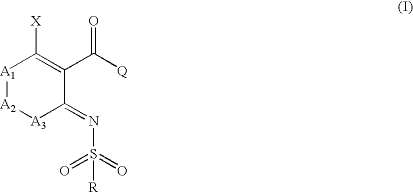

wherein A₁, A₂, A₃, R and Q are as defined above and X is chlorine or OSO₂R, by reacting a compound of formula Ia

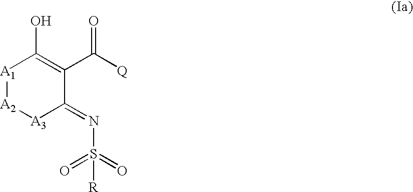

or a compound of formula II

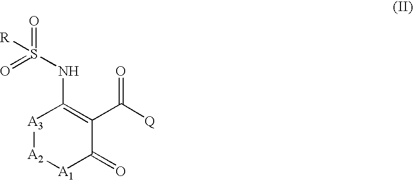

or a mixture thereof with a chlorinating agent, for example with oxalyl chloride or thionyl chloride, or with a sulfonic acid chloride of formula V, as shown in Scheme 2:

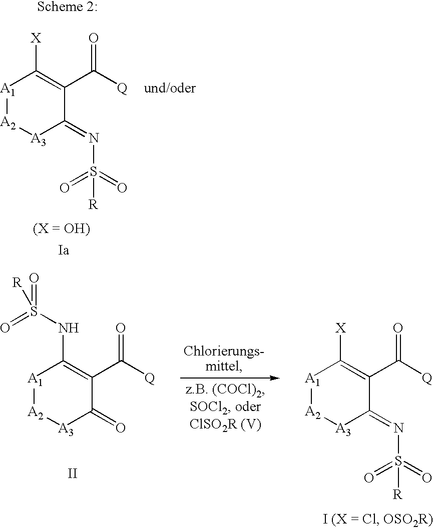

Or, for the preparation of a compound of formula I

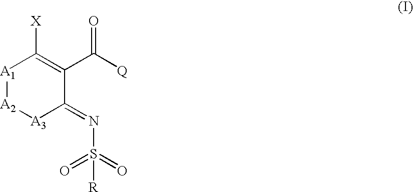

wherein X is S(O)ₙR₁, by reacting a compound of formula I wherein X is chlorine or OSO₂R and A₁, A₂, A₃, R, R₁, n and Q are as defined above, in the presence of a base, for example triethylamine, sodium hydride or potassium carbonate, with a thio compound of formula VI

HSR₁ (VI)

or with a salt thereof to form a compound of formula I

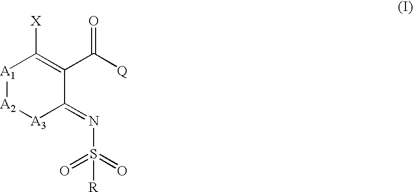

wherein A₁, A₂, A₃, R, R₁ and Q are as defined above and n is 0, and then, if m is 1 or 2, treating that compound with an oxidising agent, as shown in Scheme 3:

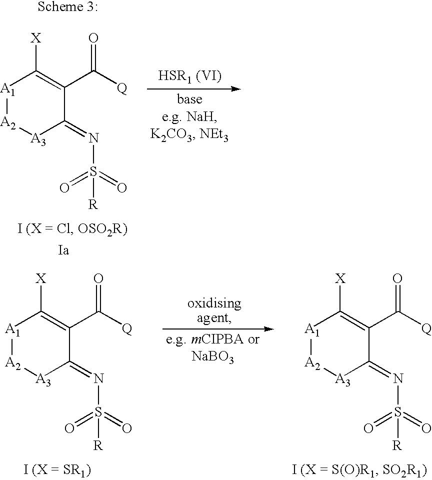

Or, for the preparation of a compound of formula I or Ia

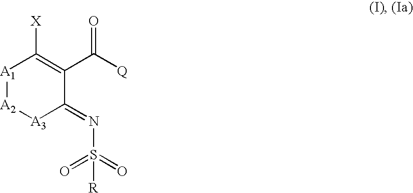

wherein A₁, A₂, A₃, R and Q are as defined above and X is hydroxy, chlorine or OSO₂R, by reacting a compound of formula VII

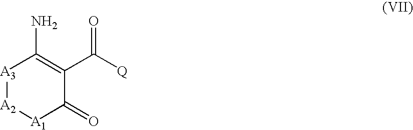

wherein A₁, A₂, A₃ and Q are as defined above, in the presence of a suitable base, for example sodium hydride or lithium hexamethyldisilazane, with a corresponding sulfonic acid chloride of formula V

CISO₂R (V)

and thus, depending on the amount of sulfonic acid chloride of formula V used and on the amount of base used, either obtaining directly a compound of formula I

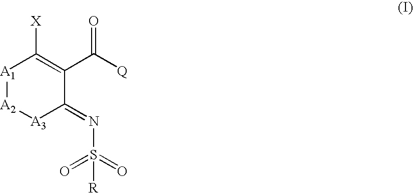

wherein X is chlorine or OSO₂R, or obtaining a compound of formula I wherein X is hydroxy (that is to say a compound of formula Ia)

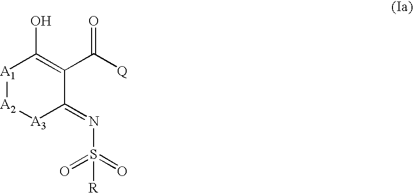

and/or a compound of formula II

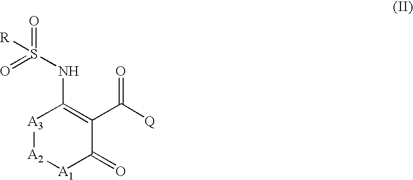

in each of which A₁₃ A₂, A₃, R and Q are as defined above, and then treating that compound further, as indicated above (see Scheme 2), with a corresponding chlorinating agent. This is shown in Scheme 4:

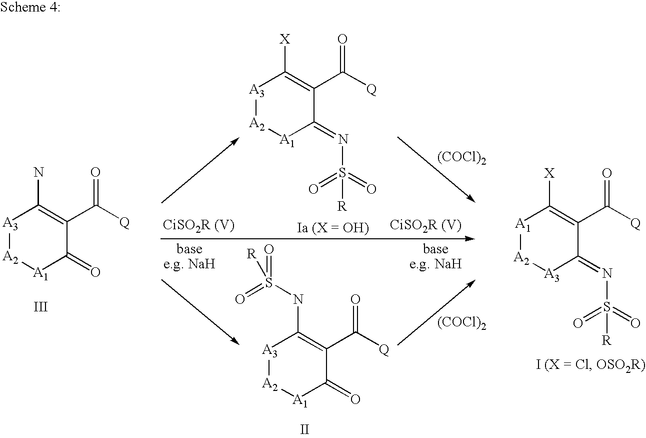

The compounds of formula Ia are valuable intermediates for the preparation of the compounds of formula I. By means of those intermediates, the compounds of formula I can be prepared in an especially simple manner and in good yields and with high quality. The present invention therefore relates also to the use of the compounds of formula Ia in the preparation of the compounds of formula I.

In addition, compounds of formula Ia are likewise distinguished by pronounced herbicidal properties.

The compounds of formula Ia

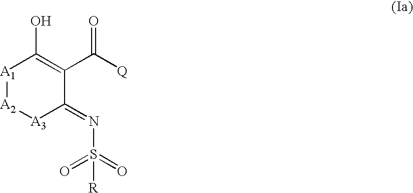

wherein A₁, A₂, A₃, R and Q are as defined for formula I, can be prepared, for example, by isomerising a compound of formula II

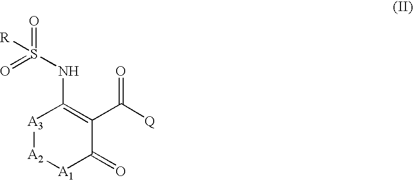

wherein A₁, A₂, A₃, R and Q are as defined above, in the presence of a catalytic or stoichiometric amount of base, for example triethylamine or sodium hydride, or in the presence of a catalytic amount of acid, for example acetic acid, formic acid, p-toluenesulfonic acid or trifluoroacetic acid. This is shown in Scheme 5:

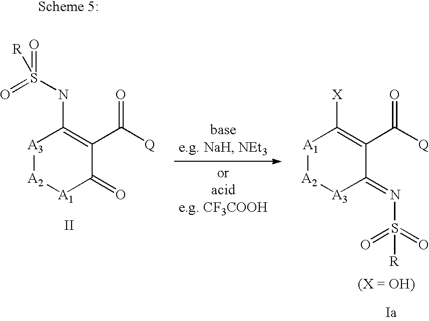

Compounds of formula Ia can also be prepared by reacting a corresponding chlorine compound of formula III

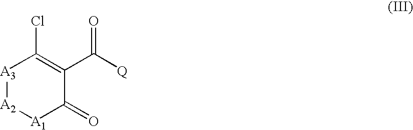

wherein A₁, A₂, A₃ and Q are as defined for formula I, with a sulfonamide of formula IV

NH₂SO₂R (IV),

which can be prepared by aminolysis from the corresponding sulfonic acid chloride V

CISO₂R (V),

or with a salt IVa thereof

M⁺⁻NHSO₂R (IVa)

(e.g. a lithium or sodium salt), thus yielding a compound of formula Ia, or a compound of formula II

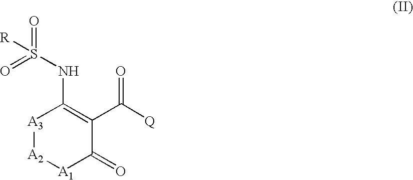

wherein A₁, A₂, A₃, R and Q are as defined above, or a compound of formula Ia in admixture with a compound of formula II, as shown in Scheme 1:

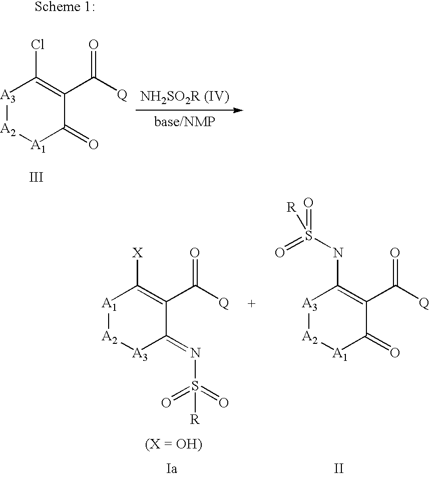

The compounds of formula II used as starting materials can likewise be prepared by methods which are generally known per se. For example, the compounds of formula II can be prepared by

a) converting a hydroxy compound of formula VIII

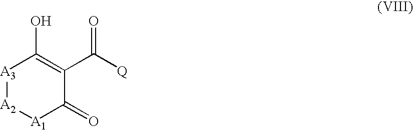

wherein A₁, A₂, A₃ and Q are as defined above, into a chlorine compound of formula III

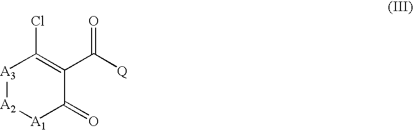

wherein A₁, A₂, A₃ and Q are as defined above, by means of a chlorinating agent, such as oxalyl chloride or thionyl chloride, and then reacting the compound of formula III with a sulfonamide of formula IV or with a salt thereof; or

b) treating the chlorine compound of formula III obtained from a hydroxy compound of formula VII with ammonia, and then reacting the resulting amino preparation of formula VII

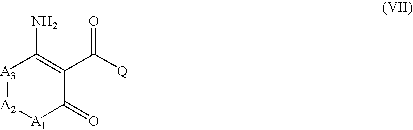

wherein A₁, A₂, A₃ and Q are as defined above, in the presence of a strong base (e.g. LDA, lithium hexamethyldisilazane or sodium hydride), with the corresponding sulfonic acid chloride (V). These reactions are shown in Scheme 6:

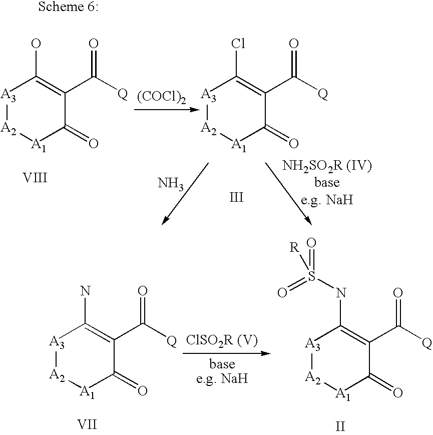

The compounds of formulae III and VII are either known or can be prepared analogously to known processes, for example the processes described in EP-A-0 249 813, DE 4 241 999, WO 00/15615 and WO 00/39094. The hydroxy compounds of formula VIII used as starting materials are either known or can be prepared according to generally described methods, for example as described in the above-mentioned citations.

According to Reaction Schemes 2 and 5, the preparation of the compounds of formula I and III is carried out using a chlorinating agent, for example thionyl chloride, phosphorus penta-chloride or phosphorus oxychloride or, preferably, oxalyl chloride. The reaction is preferably carried out in an inert organic solvent, for example in an aliphatic, halogenated aliphatic, aromatic or halogenated aromatic hydrocarbon, for example n-hexane, benzene, toluene, xylene, dichloromethane, 1,2-dichloroethane or chlorobenzene, at reaction temperatures in the range from −20° C. to the reflux temperature of the reaction mixture, preferably at approximately from 40 to 100° C., and in the presence of a catalytic amount of N,N-dimethyl-formamide.

The end products of formula I can be isolated in conventional manner by concentration or evaporation of the solvent, and can be purified by recrystallisation or trituration of the solid residue in solvents in which they are not readily soluble, such as ethers, aromatic hydrocarbons or chlorinated hydrocarbons, by distillation or by means of column chromatography or by means of an HPLC technique using a suitable eluant.

The person skilled in the art will also be familiar with the order in which the reactions should be carried out in order to avoid as far as possible secondary reactions.

Where the synthesis carried out is not targeted at the isolation of pure isomers, the product can be in the form of a mixture of two or more isomers, for example chiral centres in the case of alkyl groups, or cis/trans isomers in the case of alkenyls of the group R, R₁ or R₂, or <E> or <Z> isomers in respect of the C═NSO₂R group. All those isomers can be separated by methods known per se.

Compounds of formula I wherein Q is pyridyl N-oxide can be prepared by reacting a compound of formula I wherein Q is pyridyl with a suitable oxidising agent, for example with a H₂O₂-urea adduct, in the presence of an acid anhydride, for example trifluoroacetic anhydride. That reaction can be carried out both with compounds of formula I and at the stage of compounds of formula III or VIII.

The reactions to form compounds of formula I are advantageously carried out in aprotic, inert organic solvents. Such solvents are hydrocarbons, such as benzene, toluene, xylene or cyclohexane, chlorinated hydrocarbons, such as dichloromethane, trichloromethane, tetra-chloromethane or chlorobenzene, ethers, such as diethyl ether, ethylene glycol dimethyl ether, diethylene glycol dimethyl ether, tetrahydrofuran or dioxane, nitriles, such as aceto-nitrile or propionitrile, amides, such as N,N-dimethylformamide, diethylformamide or N-methylpyrrolidinone. The reaction temperatures are preferably from −20° C. to +120° C. If the reactions are slightly exothermic, they may generally be carried out at room temperature. In order to shorten the reaction time or in order to initiate the reaction, the reaction mixture may optionally be heated for a short time up to its boiling point. The reaction times can also be shortened by addition of suitable bases as reaction catalysts. Suitable bases are especially the tertiary amines, such as trimethylamine, triethylamine, quinuclidine, 1,4-diazabicyclo-[2.2.2]octane, 1,5-diazabicyclo[4.3.0]non-5-ene or 1,5-diazabicyclo[5.4.0]undec-7-ene. However, it is also possible to use as bases inorganic bases, such as hydrides, for example sodium or calcium hydride, hydroxides, for example dry sodium or potassium hydroxide, carbonates, for example sodium and potassium carbonate, or hydrogen carbonates, for example sodium and potassium hydrogen carbonate.

For the use according to the invention of the compounds of formula I, or of compositions comprising them, there come into consideration all methods of application customary in agriculture, for example pre-emergence application, post-emergence application and seed dressing, and also various methods and techniques such as, for example, the controlled release of active ingredient. For that purpose a solution of the active ingredient is applied to mineral granule carriers or polymerised granules (urea/formaldehyde) and dried. If required, it is also possible to apply a coating (coated granules), which allows the active ingredient to be released in metered amounts over a specific period of time.

The compounds of formula I may be used as herbicides in their unmodified form, that is to say as obtained in the synthesis, but they are preferably formulated in customary manner together with the adjuvants conventionally employed in formulation technology, for example into emulsifiable concentrates, directly sprayable or dilutable solutions, dilute emulsions, wettable powders, soluble powders, dusts, granules or microcapsules. Such formulations are described, for example, on pages 9 to 13 of WO 97/34485. As with the nature of the compositions, the methods of application, such as spraying, atomising, dusting, wetting, scattering or pouring, are chosen in accordance with the intended objectives and the prevailing circumstances.

The formulations, that is to say the compositions, preparations or mixtures comprising the compound (active ingredient) of formula I or at least one compound of formula I and, usually, one or more solid or liquid formulation adjuvants, are prepared in known manner, e.g. by homogeneously mixing and/or grinding the active ingredients with the formulation adjuvants, for example solvents or solid carriers. Surface-active compounds (surfactants) may additionally also be used in the preparation of the formulations. Examples of solvents and solid carriers are given, for example, on page 6 of WO 97/34485.

Depending upon the nature of the compound of formula I to be formulated, suitable surface-active compounds are non-ionic, cationic and/or anionic surfactants and surfactant mixtures having good emulsifying, dispersing and wetting properties.

Examples of suitable anionic, non-ionic and cationic surfactants are listed, for example, on pages 7 and 8 of WO 97/34485.

In addition, the surfactants conventionally employed in formulation technology, which are described, inter alia, in “McCutcheon's Detergents and Emulsifiers Annual” MC Publishing Corp., Ridgewood N.J., 1981, Stache, H., “Tensid-Taschenbuch”, Carl Hanser Verlag, Munich/Vienna, 1981, and M. and J. Ash, “Encyclopedia of Surfactants”, Vol. I-III, Chemical Publishing Co., New York, 1980-81, are also suitable for the preparation of the herbicidal compositions according to the invention.

The herbicidal formulations generally comprise from 0.1 to 99% by weight, especially from 0.1 to 95% by weight, of herbicide, from 1 to 99.9% by weight, especially from 5 to 99.8% by weight, of a solid or liquid formulation adjuvant, and from 0 to 25% by weight, especially from 0.1 to 25% by weight, of a surfactant. Whereas commercial products will preferably be formulated as concentrates, the end user will normally employ dilute formulations. The compositions may also comprise further ingredients, such as stabilisers, for example vegetable oils or epoxidised vegetable oils (epoxidised coconut oil, rapeseed oil or soybean oil), anti-foams, for example silicone oil, preservatives, viscosity regulators, binders, tackifiers, and also fertilisers or other active ingredients.

The compounds of formula I are generally applied to plants or the locus thereof at rates of application of from 0.001 to 4 kg/ha, especially from 0.005 to 2 kg/ha. The concentration required to achieve the desired effect can be determined by experiment. It is dependent on the nature of the action, the stage of development of the cultivated plant and of the weed and on the application (place, time, method) and may vary within wide limits as a function of those parameters.

The compounds of formula I are distinguished by herbicidal and growth-inhibiting properties, allowing them to be used in crops of useful plants, especially cereals, cotton, soybeans, sugar beet, sugar cane, plantation crops, rape, maize and rice, and also for non-selective weed control. The term “crops” is to be understood as including also crops that have been made tolerant to herbicides or classes of herbicides as a result of conventional methods of breeding or genetic engineering techniques. The weeds to be controlled may be either monocotyledonous or dicotyledonous weeds, such as, for example, Stellaria, Nasturtium, Agrostis, Digitaria, Avena, Setaria, Sinapis, Lolium, Solanum, Echinochloa, Scirpus, Monochoria, Sagittaria, Bromus, Alopecurus, Sorghum halepense, Rottboellia, Cyperus, Abutilon, Sida, Xanthium, Amaranthus, Chenopodium, Ipomoea, Chrysanthemum, Galium, Viola and Veronica.

The following Examples further illustrate but do not limit the invention.

### PREPARATION EXAMPLES

### EXAMPLE P1

### Preparation of N-[3-chloro-4,4,6,6-tetramethyl-2-(2-methyl-6-trifluoromethyl-pyridine-3-carbonyl)-5-oxo-cyclohex-2-en-ylidene]-methanesulfonamide and N-[5-hydroxy-3-oxo-4,4,6,6-tetramethyl-2-(2-methyl-6-trifluoromethyl-pyridine-3-carbonyl)-cyclohex-2-en-ylidene]-methanesulfonamide:

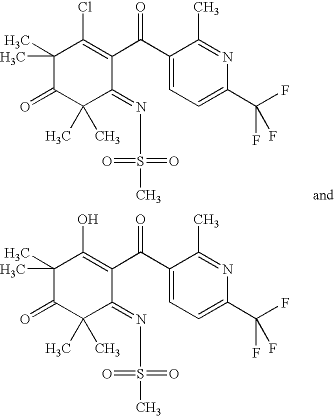

0.92 g (2.5 mmol) of 5-amino-2,2,6,6-tetramethyl-4-(2-methyl-6-trifluoromethyl-pyridine-3-carbonyl)-cyclohex-4-ene-1,3-dione (m.p. 141.5-142.5° C., prepared by treating 5-chloro-2,2,6,6-tetramethyl-4-(2-methyl-6-trifluoromethyl-pyridine-3-carbonyl)-cyclohex-4-ene-1,3-dione (compound A2-B1105, Preparation Example P6 from WO 00/39094) with 25% ammonia solution at room temperature in acetonitrile) is placed in 5 ml of anhydrous dimethylformamide. Then 0.21 g (4.8 mmol) of 55% sodium hydride dispersion in oil is stirred in, in portions. After stirring for 30 minutes, 0.29 g (3.8 mmol) of methane sulfochloride is added dropwise and stirring is carried out for a further 45 minutes. The mixture is then acidified with 1 N hydrochloric acid and extracted with ethyl acetate in the presence of a small amount of sodium chloride solution. By means of a preparative HPLC technique, silica gel Si60, 12μ, eluant gradient from 35 to 100% ethyl acetate in hexane, there are eluted, as the 1st fraction, N-[3-chloro-4,4,6,6-tetramethyl-2-(2-methyl-6-trifluoro-methyl-pyridine-3-carbonyl)-5-oxo-cyclohex-2-en-ylidene]-methanesulfonamide having a melting point of 137-138° C. and, as the 2nd fraction, N-[5-hydroxy-3-oxo-4,4,6,6-tetramethyl-2-(2-methyl-6-trifluoromethyl-pyridine-3-carbonyl)-cyclohex-2-en-ylidene]-methanesulfon-amide having a melting point of 134.5-136° C. (from diethyl ether/hexane).

### Example P2

### N-[5-Hydroxy-3-oxo-4,4,6,6-tetramethyl-2-(2-methyl-6-trifluoromethyl-pyridine-3-carbonyl)-cyclohex-2-en-ylidene]-methanesulfonamide and N-[4,4,6,6-tetramethyl-2-(2-methyl-6-difluoromethyl-pyridine-3-carbonyl)-3,5-dioxo-cyclohex-1-enyl]-phenylsulfonamide

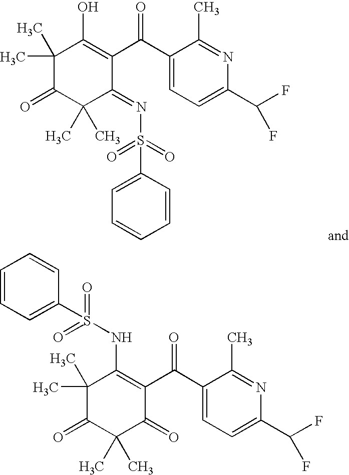

Approximately 0.34 g (7.9 mmol) of sodium hydride is placed in 5 ml of dry N-methyl-pyrrolidone. 0.62 g (3.8 mmol) of phenylsulfonamide is then introduced and the mixture is heated briefly at a temperature of 50° C. The reaction mixture is then cooled to room temperature, and 5-chloro-2,2,6,6-tetramethyl-4-(2-methyl-6-difluoromethyl-pyridine-3-carbonyl)-cyclohex-4-ene-1,3-dione, m.p. 129.5-130° C., which has been prepared analogously to Preparation Example P6 of WO 00/39094, is added, and then heating is carried out again for approximately one hour at a temperature of 50° C. The mixture is then taken up in diethyl ether and washed once with dilute hydrochloric acid and once with saturated sodium chloride solution. The residue obtained after concentration by evaporation is purified by means of column chromatography and the resulting product is recrystallised from dichloromethane and ethyl acetate (1:9). A 4:1 mixture of N-[3-hydroxy-5-oxo-4,4,6,6-tetramethyl-2-(2-methyl-6-difluoromethyl-pyridine-3-carbonyl)-cyclohex-2-en-ylidene]-phenylsulfonamide and N-[4,4,6,6-tetramethyl-2-(2-methyl-6-difluoromethyl-pyridine-3-carbonyl)-3,5-dioxo-cyclohex-1-enyl]-phenylsulfonamide is obtained in the form of an amorphous product.

### Example P3

### Preparation of N-[3-chloro-3-oxo-4,4,6,6-tetramethyl-2-(2-methyl-6-trifluoro-methyl-pyridine-3-carbonyl)-cyclohex-2-en-ylidene]-vinylsulfonamide

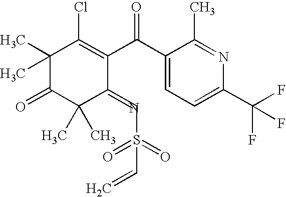

0.1 g of a 3:1 mixture of N-[5-hydroxy-3-oxo-4,4,6,6-tetramethyl-2-(2-methyl-6-trifluoro-methyl-pyridine-3-carbonyl)-cyclohex-2-en-ylidene]-methanesulfonamide (a compound of formula Ia) and N-[4,4,6,6-tetramethyl-2-(2-methyl-6-trifluoromethyl-pyridine-3-carbonyl)-3,5-dioxo-cyclohex-1-enyl]-methanesulfonamide (a compound of formula II), which has been prepared analogously to Example P2, is heated to a temperature of 40° C. with 0.03 ml of oxalyl chloride, in the presence of 3 ml of dichloromethane and a catalytic amount of dimethylformamide. After approximately 6 hours, the mixture is taken up in diethyl ether and washed once with saturated sodium chloride solution. The residue obtained after concentration by evaporation is purified by means of HPLC, yielding 0.55 g of N-[3-chloro-3-oxo-4,4,6,6-tetramethyl-2-(2-methyl-6-trifluoromethyl-pyridine-3-carbonyl)-cyclohex-2-en-ylidene]-vinylsulfonamide in the form of a resinous product which is pure according to ¹H-NMR.

### Example P4

### Preparation of N-[3-methylthio-5-oxo-4,4,6,6-tetramethyl-2-(2-methyl-6-trifluoromethyl-pyridine-3-carbonyl)-cyclohex-2-en-ylidene]-3-(2′-fluoropyridyl)sulfonamide

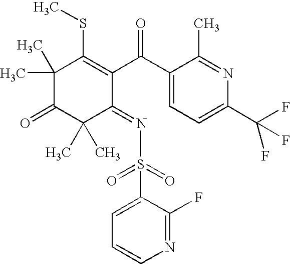

0.23 g (0.41 mmol) of N-[3-chloro-5-oxo-4,4,6,6-tetramethyl-2-(2-methyl-6-trifluoromethyl-pyridine-3-carbonyl)-cyclohex-2-en-ylidene]-3-(2′-fluoropyridyl)sulfonamide, which has been prepared analogously to Example P3, is stirred at room temperature, in the presence of 3 ml of acetonitrile, with 0.04 g (0.57 mmol) of methane thiolate. After 5 hours, the mixture is filtered over a small amount of silica gel, and the product obtained by concentration by evaporation is purified by means of an HPLC technique. Crystallisation from dichloro-methane/hexane (5:19) yields pure N-[3-methylthio-5-oxo-4,4,6,6-tetramethyl-2-(2-methyl-6-trifluoromethyl-pyridine-3-carbonyl)-cyclohex-2-en-ylidene]-3-(2′-fluoropyridyl)sulfonamide having a melting point of 133.4-134.4° C.

All the other compounds of formula I can be prepared analogously to the above descriptions and examples of preparation.

TABLE 1
Compounds of formula I:

| | |
|---|---|
| | I |
| 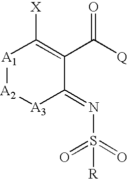 | |

| Comp. | | | | | | | Phys. |
|---|---|---|---|---|---|---|---|
| No. | A₂ | A₁ | A₃ | Q | R | X | data |
| 1.001 | C(O) | C(CH₃)₂ | C(CH₃)₂ | 2-CH₃-6-CF₃-pyridyl | CH₃ | Cl | 137-138° C. |
| (P1) | | | | | | | |
| 1.002 | C(O) | C(CH₃)₂ | C(CH₃)₂ | 2-CH₃-6-CF₃-pyridyl | CH₂C(CH₃)═CH₂ | Cl | |
| 1.003 | C(O) | C(CH₃)₂ | C(CH₃)₂ | 2-CH₃OCH₂-6-CF₃-pyridyl | CH₃ | Cl | |
| 1.004 | C(O) | C(CH₃)₂ | C(CH₃)₂ | 2-CH₂OCH₂-6-CF₃-pyridyl | CH₂C(CH₃)═CH₂ | Cl | |
| 1.005 | CH₂ | C(CH₃)₂ | C(CH₃)₂ | 2-CH₃-6-CF₃-pyridyl | CH₃ | Cl | |
| 1.006 | C(O) | C(CH₃)₂ | C(CH₃)₂ | 2-CH₃-6-CF₃-pyridyl | CF₃ | Cl | |
| 1.007 | C(O) | C(CH₃)₂ | C(CH₃)₂ | 2-CH₃-6-CF₃-pyridyl | CH₂CH₃ | Cl | |
| 1.008 | CH₂ | CH₂ | CH₂ | 2-CH₃-6-CF₃-pyridyl | CH₃ | Cl | |
| 1.009 | CH₂ | C(CH₃)₂ | CH₂ | 2-CH₃-6-CF₃-pyridyl | CH₃ | Cl | |
| 1.010 | CHCH₃ | CH₂ | CH₂ | 2-CH₃-6-CF₃-pyridyl | CH₃ | Cl | |
| 1.011 | C(CH₃)₂ | CH₂ | CH₂ | 2-CH₃-6-CF₃-pyridyl | CH₃ | Cl | |
| 1.012 | CH₂ | CH(CH₂CH₂)CH | | 2-CH₃-6-CF₃-pyridyl | CH₃ | Cl | |
| 1.013 | CH₂ | CH(CH₂CH₂)CH | | 2-CH₃-6-CF₃-pyridyl | CH₂CH₃ | Cl | |
| 1.014 | CH₂ | CH(CH₂CH₂)CH | | 2-CH₃-6-CF₃-pyridyl | n-propyl | Cl | |
| 1.015 | CH₂ | CH(CH₂CH₂)CH | | 2-CH₃-6-CF₃-pyridyl | n-butyl | Cl | |
| 1.016 | CH₂ | CH(CH₂CH₂)CH | | 2-CH₃-6-CF₃-pyridyl | CH₂C(CH₃)═CH₂ | Cl | |
| 1.017 | CH₂ | CH(CH₂CH₂)CH | | 2-CH₃-6-CF₃-pyridyl | CF₃ | Cl | |
| 1.018 | CH₂ | CH(CH₂CH₂)CH | | 2-NO₂-4-SO₂CH₃-phenyl | CF₃ | Cl | |
| 1.019 | CH₂ | CH(CH₂CH₂)CH | | 2-CH₂-3-OCH₃-4-SO₂CH₃-phenyl | CH₃ | Cl | |
| 1.020 | CH₂ | CH(CH₂CH₂)CH | | 2-Cl-3-OCH₃-4-SO₂CH₃-phenyl | CH₃ | Cl | |
| 1.021 | CHCH₃ | CH₂ | CH₂ | 2-Cl-3-OCH₃-4-SO₂CH₃-phenyl | CH₃ | Cl | |
| 1.022 | CH₂ | CH₂ | CH₂ | 2-NO₂-4-SO₂CH₃-phenyl | CH₃ | Cl | |
| 1.023 | CH(CH₃) | CH₂ | CH₂ | 2-NO₂-4-SO₂CH₃-phenyl | CH₃ | Cl | |
| 1.024 | C(O) | C(CH₃)₂ | C(CH₃)₂ | 2-NO₂-4-CN-phenyl | CH₃ | Cl | |
| 1.025 | C(O) | C(CH₃)₂ | C(CH₃)₂ | 2-CH₃-6-CF₃-pyridyl | 2-(3′-COOMe)-pyridyl | Cl | |
| 1.026 | C(O) | C(CH₃)₂ | C(CH₃)₂ | 2-CH₃-6-CF₃-pyridyl | 3-(2′-F)-pyridyl | Cl | 175-186° C. |
| 1.027 | C(O) | C(CH₃)₂ | C(CH₃)₂ | 2-CH₃-6-CF₃-pyridyl | phenyl | Cl | 153-154° C. |
| 1.028 | C(O) | C(CH₃)₂ | C(CH₃)₂ | 2-CH₃-6-CF₃-pyridyl | vinyl | Cl | 138-140° C. |
| (P3) | | | | | | | |
| 1.029 | C(O) | C(CH₃)₂ | C(CH₃)₂ | 2-CH₃-6-CF₃-pyridyl | CH₂Cl | Cl | |
| 1.030 | C(O) | C(CH₃)₂ | C(CH₃)₂ | 2-CH₃-6-CF₃-pyridyl | 3-(2′-Cl)-pyridyl | Cl | 168-170° C. |
| 1.031 | C(O) | C(CH₃)₂ | C(CH₃)₂ | 2-CH₃-6-CHF₂-pyridyl | phenyl | Cl | 155.5-156.5° C. |
| 1.032 | C(O) | C(CH₃)₂ | C(CH₃)₂ | 2-CH₃-6-CF₃-pyridyl | p-tolyl | Cl | |
| 1.033 | C(O) | C(CH₃)₂ | C(CH₃)₂ | 2-CH₃-6-CF₃-pyridyl | 4-OMe-phenyl | Cl | |
| 1.034 | C(O) | C(CH₃)₂ | C(CH₃)₂ | 2-CH₃-6-CF₃-pyridyl | 4-Cl-phenyl | Cl | |
| 1.035 | C(O) | C(CH₃)₂ | C(CH₃)₂ | 2-CH₃-6-CF₃-pyridyl | 3-CF₃-phenyl | Cl | |
| 1.036 | C(O) | C(CH₃)₂ | C(CH₃)₂ | 2-CH₃-6-CF₃-pyridyl | 2-Cl-phenyl | Cl | |
| 1.037 | C(O) | C(CH₃)₂ | C(CH₃)₂ | 2-CH₃-6-CF₃-pyridyl | 2-(ClCH₂CH₂O)-phenyl | Cl | |
| 1.038 | C(O) | C(CH₃)₂ | C(CH₃)₂ | 2-CH₃-6-CF₃-pyridyl | 2-(COOMe)-phenyl | Cl | |
| 1.039 | C(O) | C(CH₃)₂ | C(CH₃)₂ | 2-CH₃-6-CF₃-pyridyl | 2-(CONMe₂)-phenyl | Cl | |
| 1.040 | C(O) | C(CH₃)₂ | C(CH₃)₂ | 2-CH₃-6-CF₃-pyridyl | 2-OCHF₂-phenyl | Cl | |
| 1.041 | C(O) | C(CH₃)₂ | C(CH₃)₂ | 2-CH₃-6-CF₃-pyridyl | 2-(3′-OCHF₂)-pyridyl | Cl | |
| 1.042 | C(O) | C(CH₃)₂ | C(CH₃)₂ | 2-CH₃-6-CF₃-pyridyl | 2-(3′-F)-pyridyl | Cl | |
| 1.043 | C(O) | C(CH₃)₂ | C(CH₃)₂ | 2-CH₃-6-CF₃-pyridyl | 3-(2′-COOMe)-thienyl | Cl | |
| 1.044 | C(O) | C(CH₃)₂ | C(CH₃)₂ | 2-CH₃-6-CF₃-pyridyl | 2-(5′-Cl)-thienyl | Cl | |
| 1.045 | C(O) | C(CH₃)₂ | C(CH₃)₂ | 2-CH₃-6-CF₃-pyridyl | 2-(3′-CONMe₂)-pyridyl | Cl | |
| 1.046 | C(O) | C(CH₃)₂ | C(CH₃)₂ | 2-CH₃-6-CF₃-pyridyl | N(CH₃)₂ | Cl | |
| 1.047 | C(O) | C(CH₃)₂ | C(CH₃)₂ | 2-CH₃-6-CF₃-pyridyl | C(CH₃)₂ | Cl | |
| 1.048 | C(O) | C(CH₃)₂ | C(CH₃)₂ | 2-CH₃-6-CF₃-pyridyl | CH₂CH₂CH₂CH₃ | Cl | |
| 1.049 | C(O) | C(CH₃)₂ | C(CH₃)₂ | 2-CH₃-6-CF₃-pyridyl | n-octyl | Cl | |
| 1.050 | C(O) | C(CH₃)₂ | C(CH₃)₂ | 2-CH₃-6-CF₃-pyridyl | CHCl₂ | Cl | |
| 1.051 | C(O) | C(CH₃)₂ | C(CH₃)₂ | 2-CH₃-6-CF₃-pyridyl | CH₂CH₂CH₂Cl | Cl | |
| 1.052 | C(O) | C(CH₃)₂ | C(CH₃)₂ | 2-CH₃-6-CF₃-pyridyl | benzyl | Cl | |
| 1.053 | C(O) | C(CH₃)₂ | C(CH₃)₂ | 2-CH₃-6-CF₃-pyridyl | CH₃ | SCH₃ | |
| 1.054 | C(O) | C(CH₃)₂ | C(CH₃)₂ | 2-CH₃-6-CF₃-pyridyl | CH₂C(CH₃)═CH₂ | SCH₃ | |
| 1.055 | C(O) | C(CH₃)₂ | C(CH₃)₂ | 2-CH₃OCH₂-6-CF₃-pyridyl | CH₃ | SCH₃ | |
| 1.056 | C(O) | C(CH₃)₂ | C(CH₃)₂ | 2-CH₂OCH₂-6-CF₃-pyridyl | CH₂C(CH₃)═CH₂ | SCH₃ | |
| 1.057 | CH₂ | C(CH₃)₂ | C(CH₃)₂ | 2-CH₃-6-CF₃-pyridyl | CH₃ | SCH₃ | |
| 1.058 | C(O) | C(CH₃)₂ | C(CH₃)₂ | 2-CH₃-6-CF₃-pyridyl | CF₃ | SCH₃ | |
| 1.059 | C(O) | C(CH₃)₂ | C(CH₃)₂ | 2-CH₃-6-CF₃-pyridyl | CH₂CH₃ | SCH₃ | |
| 1.060 | CH₂ | CH₂ | CH₂ | 2-CH₃-6-CF₃-pyridyl | CH₃ | SCH₃ | |
| 1.061 | CH₂ | C(CH₃)₂ | CH₂ | 2-CH₃-6-CF₃-pyridyl | CH₃ | SCH₃ | |
| 1.062 | CHCH₃ | CH₂ | CH₂ | 2-CH₃-6-CF₃-pyridyl | CH₃ | SCH₃ | |
| 1.063 | C(CH₃)₂ | CH₂ | CH₂ | 2-CH₃-6-CF₃-pyridyl | CH₃ | SCH₃ | |
| 1.064 | CH₂ | CH(CH₂CH₂)CH | | 2-CH₃-6-CF₃-pyridyl | CH₃ | SCH₃ | |
| 1.065 | CH₂ | CH(CH₂CH₂)CH | | 2-CH₃-6-CF₃-pyridyl | CH₂CH₃ | SCH₃ | |
| 1.066 | CH₂ | CH(CH₂CH₂)CH | | 2-CH₃-6-CF₃-pyridyl | n-propyl | SCH₃ | |
| 1.067 | CH₂ | CH(CH₂CH₂)CH | | 2-CH₃-6-CF₃-pyridyl | n-butyl | SCH₃ | |
| 1.068 | CH₂ | CH(CH₂CH₂)CH | | 2-CH₃-6-CF₃-pyridyl | CH₂C(CH₃)═CH₂ | SCH₃ | |
| 1.069 | CH₂ | CH(CH₂CH₂)CH | | 2-CH₃-6-CF₃-pyridyl | CF₃ | SCH₃ | |
| 1.070 | CH₂ | CH(CH₂CH₂)CH | | 2-NO₂-4-SO₂CH₃-phenyl | CF₃ | SCH₃ | |
| 1.071 | CH₂ | CH(CH₂CH₂)CH | | 2-CH₂-3-OCH₃-4-SO₂CH₃-phenyl | CH₃ | SCH₃ | |
| 1.072 | CH₂ | CH(CH₂CH₂)CH | | 2-Cl-3-OCH₃-4-SO₂CH₃-phenyl | CH₃ | SCH₃ | |
| 1.073 | CHCH₃ | CH₂ | CH₂ | 2-Cl-3-OCH₃-4-SO₂CH₃-phenyl | CH₃ | SCH₃ | |
| 1.074 | CH₂ | CH₂ | CH₂ | 2-NO₂-4-SO₂CH₃-phenyl | CH₃ | SCH₃ | |
| 1.075 | CH(CH₃) | CH₂ | CH₂ | 2-NO₂-4-SO₂CH₃-phenyl | CH₃ | SCH₃ | |
| 1.076 | C(O) | C(CH₃)₂ | C(CH₃)₂ | 2-NO₂-4-CN-phenyl | CH₃ | SCH₃ | |
| 1.077 | C(O) | C(CH₃)₂ | C(CH₃)₂ | 2-CH₃-6-CF₃-pyridyl | 2-(3′-COOMe)-pyridyl | SCH₃ | |
| 1.078 | C(O) | C(CH₃)₂ | C(CH₃)₂ | 2-CH₃-6-CF₃-pyridyl | 3-(2′-F)-pyridyl | SCH₃ | 133-135° C. |
| (P4) | | | | | | | |
| 1.079 | C(O) | C(CH₃)₂ | C(CH₃)₂ | 2-CH₃-6-CF₃-pyridyl | phenyl | SCH₃ | 143-145° C. |
| 1.080 | C(O) | C(CH₃)₂ | C(CH₃)₂ | 2-CH₃-6-CF₃-pyridyl | vinyl | SCH₃ | |
| 1.081 | C(O) | C(CH₃)₂ | C(CH₃)₂ | 2-CH₃-6-CF₃-pyridyl | CH₂Cl | SCH₃ | |
| 1.082 | C(O) | C(CH₃)₂ | C(CH₃)₂ | 2-CH₃-6-CF₃-pyridyl | 3-(2′-Cl)-pyridyl | SCH₃ | 166-167° C. |
| 1.083 | C(O) | C(CH₃)₂ | C(CH₃)₂ | 2-CH₃-6-CHF₂-pyridyl | phenyl | SCH₃ | 140-141° C. |
| 1.084 | C(O) | C(CH₃)₂ | C(CH₃)₂ | 2-CH₃-6-CF₃-pyridyl | p-tolyl | SCH₃ | |
| 1.085 | C(O) | C(CH₃)₂ | C(CH₃)₂ | 2-CH₃-6-CF₃-pyridyl | 4-OMe-phenyl | SCH₃ | |
| 1.086 | C(O) | C(CH₃)₂ | C(CH₃)₂ | 2-CH₃-6-CF₃-pyridyl | 4-Cl-phenyl | SCH₃ | |
| 1.087 | C(O) | C(CH₃)₂ | C(CH₃)₂ | 2-CH₃-6-CF₃-pyridyl | 3-CF₃-phenyl | SCH₃ | |
| 1.088 | C(O) | C(CH₃)₂ | C(CH₃)₂ | 2-CH₃-6-CF₃-pyridyl | 2-Cl-phenyl | SCH₃ | |
| 1.089 | C(O) | C(CH₃)₂ | C(CH₃)₂ | 2-CH₃-6-CF₃-pyridyl | 2-(ClCH₂CH₂O)-phenyl | SCH₃ | |
| 1.090 | C(O) | C(CH₃)₂ | C(CH₃)₂ | 2-CH₃-6-CF₃-pyridyl | 2-(COOMe)-phenyl | SCH₃ | |
| 1.091 | C(O) | C(CH₃)₂ | C(CH₃)₂ | 2-CH₃-6-CF₃-pyridyl | 2-(CONMe₂)-phenyl | SCH₃ | |
| 1.092 | C(O) | C(CH₃)₂ | C(CH₃)₂ | 2-CH₃-6-CF₃-pyridyl | 2-OCHF₂-phenyl | SCH₃ | |
| 1.093 | C(O) | C(CH₃)₂ | C(CH₃)₂ | 2-CH₃-6-CF₃-pyridyl | 2-(3′-OCHF₂)-pyridyl | SCH₃ | |
| 1.094 | C(O) | C(CH₃)₂ | C(CH₃)₂ | 2-CH₃-6-CF₃-pyridyl | 2-(3′-F)-pyridyl | SCH₃ | |
| 1.095 | C(O) | C(CH₃)₂ | C(CH₃)₂ | 2-CH₃-6-CF₃-pyridyl | 3-(2′-COOMe)-thienyl | SCH₃ | |
| 1.096 | C(O) | C(CH₃)₂ | C(CH₃)₂ | 2-CH₃-6-CF₃-pyridyl | 2-(5′-Cl)-thienyl | SCH₃ | |
| 1.097 | C(O) | C(CH₃)₂ | C(CH₃)₂ | 2-CH₃-6-CF₃-pyridyl | 2-(3′-CONMe₂)-pyridyl | SCH₃ | |
| 1.098 | C(O) | C(CH₃)₂ | C(CH₃)₂ | 2-CH₃-6-CF₃-pyridyl | N(CH₃)₂ | SCH₃ | |
| 1.099 | C(O) | C(CH₃)₂ | C(CH₃)₂ | 2-CH₃-6-CF₃-pyridyl | C(CH₃)₂ | SCH₃ | |
| 1.100 | C(O) | C(CH₃)₂ | C(CH₃)₂ | 2-CH₃-6-CF₃-pyridyl | CH₂CH₂CH₂CH₃ | SCH₃ | |
| 1.101 | C(O) | C(CH₃)₂ | C(CH₃)₂ | 2-CH₃-6-CF₃-pyridyl | n-octyl | SCH₃ | |
| 1.102 | C(O) | C(CH₃)₂ | C(CH₃)₂ | 2-CH₃-6-CF₃-pyridyl | CHCl₂ | SCH₃ | |
| 1.103 | C(O) | C(CH₃)₂ | C(CH₃)₂ | 2-CH₃-6-CF₃-pyridyl | CH₂CH₂CH₂Cl | SCH₃ | |
| 1.104 | C(O) | C(CH₃)₂ | C(CH₃)₂ | 2-CH₃-6-CF₃-pyridyl | benzyl | SCH₃ | |
| 1.105 | C(O) | C(CH₃)₂ | C(CH₃)₂ | 2-CH₃-6-CF₃-pyridyl | phenyl | SCH₂CH₃ | |
| 1.106 | C(O) | C(CH₃)₂ | C(CH₃)₂ | 2-CH₃-6-CF₃-pyridyl | CH₃ | S-allyl | |
| 1.107 | C(O) | C(CH₃)₂ | C(CH₃)₂ | 2-CH₃-6-CF₃-pyridyl | CH₃ | S-octyl | |
| 1.108 | C(O) | C(CH₃)₂ | C(CH₃)₂ | 2-CH₃-6-CF₃-pyridyl | CH₃ | S-phenyl | |
| 1.109 | C(O) | C(CH₃)₂ | C(CH₃)₂ | 2-CH₃-6-CF₃-pyridyl | phenyl | S(O)CH₃ | |
| 1.110 | C(O) | C(CH₃)₂ | C(CH₃)₂ | 2-CH₃-6-CF₃-pyridyl | phenyl | S(O)₂CH₃ | |

TABLE 2
Compounds of formula Ia:

| | |
|---|---|
| | Ia |
| 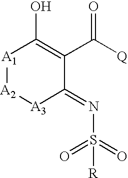 | |

| Comp. | | | | | | Phys. |
|---|---|---|---|---|---|---|
| No. | A₂ | A₁ | A₃ | Q | R | data (m.p.) |
| 2.001 | C(O) | C(CH₃)₂ | C(CH₃)₂ | 2-CH₃-6-CF₃-pyridyl | CH₃ | 134.5-136° C. |
| (P1) | | | | | | |
| 2.002 | C(O) | C(CH₃)₂ | C(CH₃)₂ | 2-CH₃-6-CF₃-pyridyl | CH₂C(CH₃)═CH₂ | resin |
| 2.003 | C(O) | C(CH₃)₂ | C(CH₃)₂ | 2-CH₂OCH₃-6-CF₃-pyridyl | CH₃ | 166-168° C. |
| 2.004 | C(O) | C(CH₃)₂ | C(CH₃)₂ | 2-CH₂OCH₃-6-CF₃-pyridyl | CH₂C(CH₃)═CH₂ | resin |
| 2.005 | CH₂ | C(CH₃)₂ | C(CH₃)₂ | 2-CH₃-6-CF₃-pyridyl | CH₃ | |
| 2.006 | C(O) | C(CH₃)₂ | C(CH₃)₂ | 2-CH₃-6-CF₃-pyridyl | CF₃ | 162-172° C. |
| | | | | | | (amorphous) |
| 2.007 | C(O) | C(CH₃)₂ | C(CH₃)₂ | 2-CH₃-6-CF₃-pyridyl | CH₂CH₃ | amorphous |
| | | | | | | crystals |
| 2.008 | CH₂ | CH₂ | CH₂ | 2-CH₃-6-CF₃-pyridyl | CH₃ | |
| 2.009 | CH₂ | C(CH₃)₂ | CH₂ | 2-CH₃-6-CF₃-pyridyl | CH₃ | |
| 2.010 | CHCH₃ | CH₂ | CH₂ | 2-CH₃-6-CF₃-pyridyl | CH₃ | |
| 2.011 | C(CH₃)₂ | CH₂ | CH₂ | 2-CH₃-6-CF₃-pyridyl | CH₃ | |
| 2.012 | CH₂ | CH(CH₂CH₂)CH | | 2-CH₃-6-CF₃-pyridyl | CH₃ | |
| 2.013 | CH₂ | CH(CH₂CH₂)CH | | 2-CH₃-6-CF₃-pyridyl | CH₂CH₃ | |
| 2.014 | CH₂ | CH(CH₂CH₂)CH | | 2-CH₃-6-CF₃-pyridyl | CH₂CH₂CH₃ | |
| 2.015 | CH₂ | CH(CH₂CH₂)CH | | 2-CH₃-6-CF₃-pyridyl | CH₂CH₂CH₂CH₃ | |
| 2.016 | CH₂ | CH(CH₂CH₂)CH | | 2-CH₃-6-CF₃-pyridyl | CH₂C(CH₃)═CH₂ | 87° C. (decomp.) |
| 2.017 | CH₂ | CH(CH₂CH₂)CH | | 2-CH₃-6-CF₃-pyridyl | CF₃ | |
| 2.018 | CH₂ | CH(CH₂CH₂)CH | | 2-NO₂-4-SO₂CH₃-phenyl | CF₃ | |
| 2.019 | CH₂ | CH(CH₂CH₂)CH | | 2-CH₃-3-OCH₃-4-SO₂CH₃-phenyl | CH₃ | |
| 2.020 | CH₂ | CH(CH₂CH₂)CH | | 2-Cl-3-OCH₃-4-SO₂CH₃-phenyl | CH₃ | |
| 2.021 | CHCH₃ | CH₂ | CH₂ | 2-Cl-3-OCH₃-4-SO₂CH₃-phenyl | CH₃ | |
| 2.022 | CH₂ | CH₂ | CH₂ | 2-NO₂-4-SO₂CH₃-phenyl | CH₃ | |
| 2.023 | C(O) | C(CH₃)₂ | C(CH₃)₂ | 2-NO₂-4-SO₂CH₃-phenyl | CH₃ | |
| 2.024 | C(O) | C(CH₃)₂ | C(CH₃)₂ | 2-NO₂-4-CN-phenyl | CH₃ | |
| 2.025 | C(O) | C(CH₃)₂ | C(CH₃)₂ | 2-CH₃-6-CF₃-pyridyl | 2-(3′-COOMe)-pyridyl | resin |
| 2.026 | C(O) | C(CH₃)₂ | C(CH₃)₂ | 2-CH₃-6-CF₃-pyridyl | 3-(2′-F)-pyridyl | 192-193° C. |
| 2.027 | C(O) | C(CH₃)₂ | C(CH₃)₂ | 2-CH₃-6-CF₃-pyridyl | phenyl | 172-174° C. |
| 2.028 | C(O) | C(CH₃)₂ | C(CH₃)₂ | 2-CH₃-6-CF₃-pyridyl | vinyl | 168-170° C. |
| 2.029 | C(O) | C(CH₃)₂ | C(CH₃)₂ | 2-CH₃-6-CF₃-pyridyl | CH₂Cl | 162-164° C. |
| 2.030 | C(O) | C(CH₃)₂ | C(CH₃)₂ | 2-CH₃-6-CF₃-pyridyl | 3-(2′-Cl)-pyridyl | 186-187° C. |
| 2.031 | C(O) | C(CH₃)₂ | C(CH₃)₂ | 2-CH₃-6-CHF₂-pyridyl | phenyl | amorphous |
| (P2) | | | | | | crystals |
| 2.032 | C(O) | C(CH₃)₂ | C(CH₃)₂ | 2-CH₃-6-CF₃-pyridyl | p-tolyl | |
| 2.033 | C(O) | C(CH₃)₂ | C(CH₃)₂ | 2-CH₃-6-CF₃-pyridyl | 4-OMe-phenyl | |
| 2.034 | C(O) | C(CH₃)₂ | C(CH₃)₂ | 2-CH₃-6-CF₃-pyridyl | 4-Cl-phenyl | |
| 2.035 | C(O) | C(CH₃)₂ | C(CH₃)₂ | 2-CH₃-6-CF₃-pyridyl | 3-CF₃-phenyl | |
| 2.036 | C(O) | C(CH₃)₂ | C(CH₃)₂ | 2-CH₃-6-CF₃-pyridyl | 2-Cl-phenyl | |
| 2.037 | C(O) | C(CH₃)₂ | C(CH₃)₂ | 2-CH₃-6-CF₃-pyridyl | 2-(ClCH₂CH₂O)-phenyl | |
| 2.038 | C(O) | C(CH₃)₂ | C(CH₃)₂ | 2-CH₃-6-CF₃-pyridyl | 2-(COOMe)-phenyl | |
| 2.039 | C(O) | C(CH₃)₂ | C(CH₃)₂ | 2-CH₃-6-CF₃-pyridyl | 2-(CONMe₂)-phenyl | |
| 2.040 | C(O) | C(CH₃)₂ | C(CH₃)₂ | 2-CH₃-6-CF₃-pyridyl | 2-OCHF₂-phenyl | |
| 2.041 | C(O) | C(CH₃)₂ | C(CH₃)₂ | 2-CH₃-6-CF₃-pyridyl | 2-(3′-OCHF₂)-pyridyl | |
| 2.042 | C(O) | C(CH₃)₂ | C(CH₃)₂ | 2-CH₃-6-CF₃-pyridyl | 2-(3′-F)-pyridyl | |
| 2.043 | C(O) | C(CH₃)₂ | C(CH₃)₂ | 2-CH₃-6-CF₃-pyridyl | 3-(2′-COOMe)-thienyl | |
| 2.044 | C(O) | C(CH₃)₂ | C(CH₃)₂ | 2-CH₃-6-CF₃-pyridyl | 2-(5′-Cl)-thienyl | |
| 2.045 | C(O) | C(CH₃)₂ | C(CH₃)₂ | 2-CH₃-6-CF₃-pyridyl | 2-(3′-CONMe₂)-pyridyl | |
| 2.046 | C(O) | C(CH₃)₂ | C(CH₃)₂ | 2-CH₃-6-CF₃-pyridyl | N(CH₃)₂ | |
| 2.047 | C(O) | C(CH₃)₂ | C(CH₃)₂ | 2-CH₃-6-CF₃-pyridyl | C(CH₃)₂ | |
| 2.048 | C(O) | C(CH₃)₂ | C(CH₃)₂ | 2-CH₃-6-CF₃-pyridyl | CH₂CH₂CH₂CH₃ | |
| 2.049 | C(O) | C(CH₃)₂ | C(CH₃)₂ | 2-CH₃-6-CF₃-pyridyl | n-octyl | |
| 2.050 | C(O) | C(CH₃)₂ | C(CH₃)₂ | 2-CH₃-6-CF₃-pyridyl | CHCl₂ | |
| 2.051 | C(O) | C(CH₃)₂ | C(CH₃)₂ | 2-CH₃-6-CF₃-pyridyl | CH₂CH₂CH₂Cl | |
| 2.052 | C(O) | C(CH₃)₂ | C(CH₃)₂ | 2-CH₃-6-CF₃-pyridyl | benzyl | |

TABLE 3
Intermediates of formula II:

| | |
|---|---|
| | (II) |
| 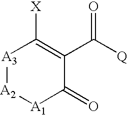 | |

| Comp. | | | | | | Phys. |
|---|---|---|---|---|---|---|
| No. | A₂ | A₁ | A₃ | Q | X | data |
| 3.001 | CH₂ | CH(CH₂—CH₂)CH | | 2-CH₃-6-CF₃-pyridyl | NH₂ | |
| 3.002 | CH₂ | CHCH₃) | CH₂ | 2-CH₃-6-CF₃-pyridyl | Cl | |
| 3.003 | CH₂ | C(CH₃)₂ | CH₂ | 2-CH₃-6-CF₃-pyridyl | Cl | |
| 3.004 | CH₂ | C(CH₃)₂ | CH₂ | 2-CH₃-6-CF₃-pyridyl | NH₂ | |
| 3.005 | C(O) | C(CH₃)₂ | C(CH₃)₂ | 2-CH₂-6-CF₃-pyridyl | NH₂ | 141.5-142.5° C. |
| 3.006 | C(O) | C(CH₃)₂ | C(CH₃)₂ | 2-CH₃-6-CHF₂-pyridyl | Cl | 129.5-130° C. |
| 3.007 | CH₂ | —CH(CH₂CH₂)CH— | | 2-CH₃-6-CF₃-pyridyl | NHSO₂CH₃ | amorphous |
| | | | | | | crystals |
| 3.008 | CH₂ | —CH(CH₂CH₂)CH— | | 2-CH₃-6-CF₃-pyridyl | NHSO₂CH₂CH₃ | 172-173° C. |
| 3.009 | CH₂ | —CH(CH₂CH₂)CH— | | 2-CH₃-6-CF₃-pyridyl | NHSO₂-n-propyl | 123-124° C. |
| 3.010 | CH₂ | —CH(CH₂CH₂)CH— | | 2-CH₃-6-CF₃-pyridyl | NHSO₂-n-butyl | 111-112° C. |
| 3.011 | CH₂ | —CH(CH₂CH₂)CH— | | 2-CH₃-6-CF₃-pyridyl | NHSO₂CH═C(CH₃)₂ | resin |
| 3.012 | C(O) | C(CH₃)₂ | C(CH₃)₂ | 2-CH₃-6-CF₃-pyridyl | SO₂NH-2-(3′-COOMe)-pyridyl | resin |

### Biological Examples

### Example B1

### Herbicidal Action Prior to Emergence of the Plants (Pre-Emergence Action)

Monocotyledonous and dicotyledonous test plants are sown in standard soil in plastic pots. Immediately after sowing, an aqueous suspension (prepared from a 25% wettable powder (Example F3, b) according to WO 97/34485) or an emulsion (prepared from a 25% emulsifiable concentrate (Example F1, c)) of the test compounds is applied by spraying at a rate of application corresponding to 250 g a.i./ha (500 litres water/ha). The test plants are then cultivated in a greenhouse under optimum conditions. After a test duration of 3 weeks the test is evaluated in accordance with a scale of nine ratings (1=total damage, 9=no action). Ratings of from 1 to 4 (especially from 1 to 3) indicate good to very good herbicidal action.

TABLE B1
Pre-emergence action of the compounds of formula I:

| Comp. | | | | | | | | |
|---|---|---|---|---|---|---|---|---|
| No. | Panicum | Digitaria | Echinochloa | Brachiaria | Euphoria | Abutilon | Amarantus | Chenopodium |
| 1.026 | 1 | 1 | 1 | 1 | 2 | 1 | 1 | 1 |
| 1.027 | 1 | 1 | 1 | 1 | 1 | 1 | 1 | 1 |
| 1.028 | 1 | 3 | 1 | 1 | 2 | 1 | 1 | 1 |
| 1.079 | 1 | 1 | 1 | 1 | 2 | 1 | 1 | 1 |
| 1.078 | 1 | 1 | 1 | 2 | 2 | 1 | 1 | 1 |

The same results are obtained when the compounds of formula I are formulated in accordance with Examples F2 and F4 to F8 according to WO 97/34485.

### Example B2

### Post-Emergence Herbicidal Action

Monocotyledonous and dicotyledonous test plants are raised in a greenhouse in plastic pots containing standard soil and at the 4- to 6-leaf stage are sprayed with an aqueous suspension of the test compounds of formula I (prepared from a 25% wettable powder (Example F3, b) according to WO 97/34485) or with an emulsion of the test compounds of formula I (prepared from a 25% emulsifiable concentrate (Example F1, c) according to WO 97/34485) at a rate of application corresponding to 250 g a.i./ha (500 litres water/ha). The test plants are then grown on in the greenhouse under optimum conditions. After a test duration of about 18 days, the test is evaluated in accordance with a scale of nine ratings (1=total damage, 9=no action). Ratings of from 1 to 4 (especially from 1 to 3) indicate good to very good herbicidal action. Compounds of formula I exhibit strong herbicidal action in this test.

TABLE B2
Post-emergence action of the compounds of formula I:

| Comp. | | | | | | | | |
|---|---|---|---|---|---|---|---|---|
| No. | Setaria | Panicum | Digitaria | Brachiaria | Abutilon | Amarantus | Chenopodium | Stellaria |
| 1.001 | 1 | 1 | 1 | 1 | 2 | 2 | 2 | 2 |
| 1.026 | 1 | 1 | 1 | 2 | 1 | 1 | 1 | 2 |
| 1.027 | 1 | 2 | 2 | 2 | 1 | 2 | 1 | 1 |
| 1.028 | 1 | 1 | 1 | 2 | 1 | 2 | 1 | 1 |
| 1.079 | 2 | 2 | 2 | 2 | 2 | 2 | 1 | 1 |
| 1.078 | 2 | 1 | 1 | 3 | 1 | 2 | 1 | 3 |

What is claimed is:

1. A compound of formula I 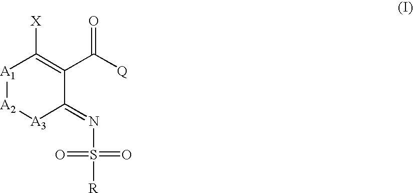
wherein
X is chlorine, OSO₂R or S(O)ₙR1;
Q is a phenyl group mono- to tetra-substituted by R₂, wherein a saturated or unsaturated 5- to 8-membered ring system may be fused to the phenyl group, which ring system may itself be mono- to tri-substituted by R₁, and may contain in the ring one, two or three hetero atom groups selected from —O—, —NR₁₀-, —S(O)ₚ— and —C(X₂)—;
or Q represents a pyridyl or pyridyl-N-oxido group mono- to tri-substituted by R₂, a pyrimidinyl group, or a 5-membered heteroaryl group mono- to tri-substituted by R₂;
A, is C(R₃R₄) or NR₂₇;
A₂ is C(R₅R₆)ₘ, C(O), oxygen, NR₇ or S(O)_{q};
A₃ is C(R₈R₉) or NR₂₈;
with the proviso that A₂ is other than NR₇ and S(O)_{q} when A₁ is NR₂₇ and/or A₃ is NR₂₈;
R is C₁-C₁₂alkyl, C₁-C₁₂haloalkyl, C₂-C₁₂alkenyl, C₂-C₁₂haloalkenyl, or is vinyl substituted by C₁-C₂alkoxycarbonyl or by phenyl, or is C₂-C₁₂alkynyl, C₂-C₁₂haloalkynyl, C₃-C₁₂allenyl, C₃-C₆cycloalkyl, NR₁₃R₁₄ or phenyl, wherein the phenyl-containing groups may themselves be mono- to penta-substituted by C₁-C₄alkyl, C₂-C₄alkenyl, C₁-C₄haloalkyl, C₁-C₄alkoxy, C₁-C₄haloalkoxy, halogen, S(O)_{f}R₁₅, S(O)₂NR₁₆R₁₇, cyano, C₁-C₄alkoxycarbonyl, C₁-C₄-alkylcarbonyl, cyclopropylcarbonyl or by nitro;
or R is C₃-C₆cycloalkyl-C₁-C₆alkyl, phenyl-C₁-C₆alkyl, hydroxy-C₁-C₁₂alkyl, C₁-C₄alkoxy-C₁-C₁₂alkyl, C₁-C₄alkylthio-C₁-C₁₂alkyl, C₁-C₄alkylsulfinyl-C₁-C₁₂alkyl, C₁-C₄alkylsulfonyl-C₁-C₁₂alkyl, cyano-C₁-C₁₂alkyl, C₁-C₆alkylcarbonyloxy-C₁-C₁₂alkyl, C₁-C₄alkoxycarbonyl-C₁-C₁₂alkyl, C₁-C₄alkoxycarbonyloxy-C₁-C₁₂alkyl, rhodano-C₁-C₁₂alkyl, benzoyloxy-C₁-C₁₂-alkyl, C₁-C₄alkylamino-C₁-C₁₂alkyl, di-(C₁-C₄alkyl)amino-C₁-C₁₂alkyl, C₁-C₁₂alkylthiocarbonyl-C₁-C₁₂alkyl or formyl-C₁-C₁₂alkyl;
or R is a five- to ten-membered monocyclic or annellated bicyclic ring system, which may be aromatic, partially saturated or fully saturated and may contain from 1 to 4 hetero atoms selected from nitrogen, oxygen and sulfur, the ring system being bonded to the sulfur atom of the S(O)₂N=group either directly or by way of a C₁-C₁₂alkylene chain, and wherein each ring system contains no more than two oxygen atoms and no more than two sulfur atoms, and wherein each ring system may itself be mono- or poly-substituted by C₁-C₆alkyl, C₁-C₆-haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, mercapto, C₁-C₆alkylthio, C₁-C₆haloalkyl-thio, C₃-C₆alkenylthio, C₃-C₆haloalkenylthio, C₃-C₆alkynylthio, C₂-C₅alkoxyalkylthio, C₃-C₅-acetylalkylthio, C₃-C₆alkoxycarbonylalkylthio, C₂-C₄cyanoalkylthio, C₁-C₆alkylsulfinyl, C₁-C₆-haloalkylsulfinyl, C₁-C₆alkylsulfonyl, C₁-C₆haloalkylsulfonyl, aminosulfonyl, C₁-C₂alkylamino-sulfonyl, di-(C₁-C₂alkyl)aminosulfonyl, di-(C₁-C₄alkyl)amino, halogen, cyano, nitro, phenylthio or by benzylthio, wherein the phenylthio and benzylthio groups may themselves be substituted on the phenyl ring by C₁-C₃alkyl, C₁-C₃haloalkyl, C₁-C₃alkoxy, C₁-C₃haloalkoxy, halogen, cyano or by nitro, and wherein the substituents on the nitrogen in the heterocyclic ringsystem cannot be halogen;
n is 0, 1 or 2;
m is 1 or 2;
p is 0, 1 or 2;
q is 0, 1 or 2;
r is 0, 1 or 2;
R₁ is C₁-C₁₂alkyl, C₁-C₁₂haloalkyl, C₃-C₁₂alkenyl, C₃-C₁₂haloalkenyl, C₃-C₁₂alkynyl, C₃-C₁₂-haloalkynyl, C₃-C₁₂allenyl, C₃-C₆cycloalkyl or phenyl, wherein the phenyl group may itself be mono- to penta-substituted by C₁-C₄alkyl, C₁-C₄haloalkyl, C₁-C₄alkoxy, C₁-C₄haloalkoxy, halogen, S(O)ᵣR₄₁, S(O)₂NR₃₉R₄₀, cyano, C₁-C₄alkoxycarbonyl or by nitro;
or R1 is phenyl-C₁-C₆alkyl, hydroxy-C₁-C₈alkyl, C₁-C₄alkoxy-C₁-C₈alkyl, C₁-C₄alkylthio-C₁-C₈-alkyl, C₃-C₆alkenylthio-C₃-C₆alkyl, cyano-C₁-C₈alkyl, C₁-C₄alkoxycarbonyl-C₁-C₈alkyl or di-(C₁-C₄alkyl)amino-C₁-C₈alkyl;
each R₂ independently is
hydrogen, halogen, C₁-C₆alkyl, C₂-C₆alkenyl, C₂-C₆haloalkenyl, C₂-C₆alkynyl, C₂-C₆halo-alkynyl, C₃-C₆cycloalkyl, C₁-C₆alkoxy, C₁-C₆haloalkoxy, C₃-C₆alkenyloxy, C₃-C₆alkynyloxy, C₁-C₆alkylthio, C₁-C₆alkylsulfinyl, C₁-C₆alkylsulfonyl, C₁-C₆haloalkyl, C₁-C₆haloalkylthio, C₁-C₆haloalkylsulfinyl, C₁-C₆haloalkylsulfonyl, C₁-C₆alkoxycarbonyl, C₁-C₆alkylcarbonyl, C₁-C₆alkylamino, di-(C₁-C₆alkyl)amino, C₁-C₆alkylaminosulfonyl, di-(C₁-C₆alkyl)aminosulfonyl, —N(R₁₈)—SO₂—R₁₉, nitro, cyano, amino, formyl, hydroxy-C₁-C₆alkyl, C₁-C₆alkoxy-C₁-C₆alkyl, C₁-C₆haloalkoxy-C₁-C₆alkyl, C₁-C₈alkylcarbonyloxy-C₁-C₄alkyl, C₁-C₆alkoxycarbonyloxy-C₁-C₆alkyl, C₁-C₆alkylthio-C₁-C₆alkyl, C₁-C₆alkylsulfinyl-C₁-C₆alkyl, C₁-C₆alkylsulfonyl-C₁-C₆-alkyl, rhodano-C₁-C₆alkyl, cyano-C₁-C₆alkyl, C₃-C₆alkenyloxy-C₁-C₃alkyl, C₃-C₆alkynyloxy-C₁-C₃alkyl, C₁-C₆alkoxy-C₁-C₆alkoxy-C₁-C₃alkyl, C₁-C₆haloalkoxy-C₁-C₆alkoxy-C₁-C₆alkyl, C₁-C₄alkoxy-C₁-C₄alkoxy-C₁-C₄alkoxy-C₁-C₃alkyl, C₁-C₄alkoxy-C₁-C₄alkoxy-C₁-C₄alkoxy-C₁-C₄alkoxy-C₁-C₃alkyl, C₁-C₆alkylcarbonyloxy-C₁-C₄alkoxy-C₁-C₄alkyl, C₁-C₆alkoxy-C₁-C₆-alkoxy, C₁-C₆alkoxycarbonyloxy-C₁-C₆alkoxy, cyano-C₁-C₆alkoxy, cyano-C₁-C₆alkenyloxy, C₁-C₆alkoxycarbonyl-C₁-C₆alkoxy-C₁-C₃alkyl, C₁-C₆alkoxycarbonyl-C₁-C₆alkoxy, C₁-C₆-alkylthio-C₁-C₆alkoxy, C₁-C₆alkylthio-C₁-C₆alkoxy-C₁-C₃alkyl, C₁-C₆alkoxycarbonyl-C₁-C₆-alkylthio, C₁-C₆alkoxycarbonyl-C₁-C₆alkylthio-C₁-C₃alkyl, C₁-C₆alkoxycarbonyl-C₁-C₆-alkylsulfinyl, C₁-C₆alkoxycarbonyl-C₁-C₆alkylsulfinyl-C₁-C₃alkyl, C₁-C₆alkoxycarbonyl-C₁-C₆-alkylsulfonyl, C₁-C₆alkoxycarbonyl-C₁-C₆alkylsulfonyl-C₁-C₃alkyl, N—(C₁-C₆alkyl)-C₁-C₆-alkylsulfonylamino-C₁-C₃alkyl, C₁-C₆alkylsulfonyloxy, C₁-C₆haloalkylsulfonyloxy, phenyl, benzyl, phenoxy, phenylthio, phenylsulfinyl, phenylsulfonyl, benzylthio, benzylsulfinyl or benzylsulfonyl, wherein the phenyl-containing groups may themselves be mono-, di- or tri-substituted by C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, C₁-C₆-alkylthio, C₁-C₆haloalkylthio, C₃-C₆alkenylthio, C₃-C₆haloalkenylthio, C₃-C₆alkynylthio, C₁-C₄-alkoxy-C₁-C₃alkylthio, C₁-C₄alkylcarbonyl-C₁-C₃alkylthio, C₁-C₄alkoxycarbonyl-C₁-C₃alkylthio, C₂-C₄cyano-C₁-C₃alkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₂alkylaminosulfonyl, di-(C₁-C₄alkyl)aminosulfonyl, C₁-C₃alkoxy-C₁-C₃alkyl, C₁-C₃alkoxycarbonyl-C₁-C₃alkyl, C₁-C₃alkylthio-C₁-C₃alkyl, alkyl-sulfinyl-C₁-C₃alkyl, alkylsulfonyl-C₁-C₃alkyl, NR₃₀R₃₁, halogen, cyano, nitro, phenyl, or by phenyl-C₁-C₃alkylene which may be interrupted by oxygen or by —S(O)ₚ—, wherein the phenyl and phenyl-C₁-C₃alkylene groups may be substituted on the phenyl ring by C₁-C₃alkyl, C₁-C₃haloalkyl, C₁-C₃alkoxy, C₁-C₃haloalkoxy, halogen, cyano or by nitro;
or each R₂ independently is C₃-C₆cycloalkyl which is bonded to the group Q either directly or by way of a C₁-C₄alkylene chain which may be interrupted by oxygen or by S(O)ₚ, wherein cycloalkyl may be substituted by C₁-C₃alkyl or by halogen, or
each R₂ independently is phenyl which is bonded to the group Q by way of a C₁-C₄alkylene chain (which may additionally be interrupted by oxygen or by S(O)p) and which may be substituted by C₁-C₃alkyl, C₁-C₃haloalkyl, C₁-C₃alkoxy, C₁-C₃haloalkoxy, halogen, cyano or by nitro;
or each R₂ independently is a five- to ten-membered monocyclic or annellated bicyclic ring system, which may be aromatic, partially saturated or fully saturated and may contain from 1 to 4 hetero atoms selected from nitrogen, oxygen and sulfur or a carbonyl group; the ring system being bonded to the group 0 either directly or by way of a C₁-C₄alkylene chain which may be interrupted by oxygen or by —S(O)ₚ—, and wherein each ring system may contain no more than two oxygen atoms and no more than two sulfur atoms, and the ring system may itself be mono-, di- or tri-substituted by halogen, C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, C₁-C₆alkylthio, C₁-C₆haloalkylthio, C₃-C₆alkenylthio, C₃-C₆haloalkenylthio, C₃-C₆alkynylthio, C₂-C₆alkoxyalkylthio, C₃-C₅acetylalkylthio, C₃-C₆alkoxycarbonylalkylthio, C₂-C₄cyanoalkylthio, C₁-C₆alkylsulfinyl, C₁-C₆haloalkylsulfinyl, C₁-C₆alkylsulfonyl, C₁-C₆haloalkylsulfonyl, aminosulfonyl, C₁-C₄alkylaminosulfonyl, C₁-C₄dialkylaminosulfonyl, R20-C₁-C₃alkylene, NR₃₂R₃₃, cyano, nitro, phenylthio or by benzylthio, wherein phenylthio and benzylthio may themselves be substituted on the phenyl ring by C₁-C₃alkyl, C₁-C₃haloalkyl, C₁-C₃alkoxy, C₁-C₃haloalkoxy, halogen, cyano or by nitro, and wherein the substituents on the nitrogen in the heterocyclic ringsystem cannot be halogen;
R₃ is hydrogen, C₁-C₆alkyl, C₂-C₆alkenyl, C₂-C₆alkynyl, C₁-C₆alkoxy, C₁-C₆alkyl-S(O)ᵣ—, C₁-C₆alkoxycarbonyl, C₁-C₆alkylcarbonyl, C₁-C₆alkyl-NHS(O)₂, C₁-C₆alkylamino, di-(C₁-C₆-alkyl)amino, hydroxy, C₁-C₆alkoxy, C₃-C₆alkenyloxy, C₃-C₆alkynyloxy, hydroxy-C₁-C₆alkyl, C₁-C₄alkylsulfonyloxy-C₁-C₆alkyl, tosyloxy-C₁-C₆alkyl, halogen, cyano, nitro, phenyl or phenyl substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, amino, C₁-C₄alkylamino, di-C₁-C₄alkylamino, C₁-C₄alkylthio, C₁-C₄-alkylsulfinyl, C₁-C₄alkylsulfonyl, C₁-C₄alkylsulfonyloxy, C₁-C₄haloalkylthio, C₁-C₄haloalkyl-sulfinyl, C₁-C₄haloalkylsulfonyl, C₁-C₄haloalkylsulfonyloxy, C₁-C₄alkylsulfonylamino, N—(C₁-C₄alkyl)-C₁-C₄alkylsulfonylamino, halogen, nitro, COOH or cyano;
R₄ is hydrogen, C₁-C₄alkyl or C₁-C₄alkyl-S(O)ᵣ—;
or R₄ together with R₃ is a C₂-C₅alkylene chain which may be interrupted by —O—, —C(O)—, —O—, —C(O)— or —S(O)ₚ—;
R₅ is hydrogen, hydroxy, C₁-C₆alkyl, C₁-C₆haloalkyl, C₁-C₄hydroxyalkyl, C₁-C₄alkoxy-C₁-C₄-alkyl, C₁-C₄alkylthio-C₁-C₄alkyl, C₁-C₄alkylthio-C₃-C₆cycloalkyl, C₁-C₄alkylcarbonyloxy-C₁-C₄-alkyl, C₁-C₄alkylsulfonyloxy-C₁-C₄alkyl, tosyloxy-C₁-C₄alkyl, di-(C₁-C₄alkoxy)-C₁-C₄alkyl, C₁-C₄alkoxycarbonyl, di-(C₁-C₃alkylthio)-C₁-C₄alkyl, (C₁-C₃alkoxy)-(C₁-C₃alkylthio)-C₁-C₄alkyl, C₃-C₅oxacycloalkyl, C₃-C₅thiacycloalkyl, C₃-C₄dioxacycloalkyl, C₃-C₄dithiacycloalkyl, C₃-C₄oxathiacycloalkyl, formyl, C₁-C₄alkoxyiminomethyl, carbamoyl, C₁-C₄alkylamino-carbonyl, di-(C₁-C₄alkyl)aminocarbonyl, phenylaminocarbonyl, benzylaminocarbonyl or phenyl, wherein the phenyl-containing groups may themselves be substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, amino, C₁-C₄alkylamino, di-C₁-C₄alkylamino, C₁-C₄alkylthio, C₁-C₄alkylsulfinyl, C₁-C₄alkyl-sulfonyl, C₁-C₄alkylsulfonyloxy, C₁-C₄haloalkylthio, C₁-C₄haloalkylsulfinyl, C₁-C₄haloalkyl-sulfonyl, C₁-C₄haloalkylsulfonyloxy, C₁-C₄alkylsulfonylamino, N—(C₁-C₄alkyl)-C₁-C₄alkyl-sulfonylamino, halogen, nitro, COOH or by cyano;
or R₅ together with R₃, R₄, R₈, R₉, R₂₇ or R₂₈ may form a direct bond or a C₁-C₄alkylene bridge, or, when m is 2, two substituents R₅ together may form a direct bond;
R₆ is hydrogen, C₁-C₄alkyl or C₁-C₄haloalkyl;
R₇ is hydrogen, C₁-C₄alkyl, C₁-C₄alkoxycarbonyl, C₁-C₄alkylcarbonyl or di-(C₁-C₄alkyl)amino-carbonyl, or phenyl which may itself be substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, C₁-C₄alkylamino, di-C₁-C₄alkylamino, C₁-C₄alkylthio, C₁-C₄alkylsulfinyl, C₁-C₄alkylsulfonyl, C₁-C₄alkylsulfonyloxy, C₁-C₄haloalkylthio, C₁-C₄haloalkylsulfinyl, C₁-C₄haloalkylsulfonyl, C₁-C₄haloalkylsulfonyloxy, C₁-C₄alkylsulfonylamino, N—(C₁-C₄alkyl)-C₁-C₄alkylsulfonylamino, halogen, nitro or by cyano;
R8 is hydrogen, C₁-C₆alkyl, C₂-C₆alkenyl, C₂-C₆alkynyl, C₁-C₆alkoxy, C₁-C₆alkyl-S(O)ᵣ— or C₁-C₆alkoxycarbonyl, C₁-C₆alkylcarbonyl, C₁-C₆alkylaminosulfonyl, C₁-C₆alkylamino, di-(C₁-C₆alkyl)amino, hydroxy, C₁-C₆alkoxy, C₃-C₆alkenyloxy, C₃-C₆alkynyloxy, hydroxy-C₁-C₆alkyl, C₁-C₄alkylsulfonyloxy-C₁-C₆alkyl, tosyloxy-C₁-C₆alkyl, halogen, cyano, nitro, phenyl, or phenyl substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, amino, C₁-C₄alkylamino, di-(C₁-C₄alkyl)amino, C₁-C₆alkylthio, C₁-C₆alkylsulfinyl, C₁-C₆alkylsulfonyl, C₁-C₄alkylsulfonyloxy, C₁-C₆haloalkyl-thio, C₁-C₆haloalkylsulfinyl, C₁-C₆haloalkylsulfonyl, C₁-C₄haloalkylsulfonyloxy, C₁-C₄alkyl-sulfonylamino, N—(C₁-C₄alkyl)-C₁-C₄alkylsulfonylamino, halogen, nitro, COOH or by cyano;
or, when A₂ is C(R₅R₆)ₘ, R₈ together with R₃ or R₄ may form a direct bond or a C₁-C₃alkylene bridge;
R₉ is hydrogen or C₁-C₄alkyl;
or R₉ together with R₈ forms a C₂-C₅alkylene bridge which may be interrupted by —O—, —C(O)—, —O—, —C(O)— or —S(O)ₚ—;
R₁₀ is hydrogen or C₁-C₆alkyl;
R₁₁ is halogen, C₁-C₆alkyl, C₁-C₆haloalkyl, C₂-C₆alkenyl, C₂-C₆haloalkenyl, C₂-C₆alkynyl, C₂-C₆haloalkynyl, C₁-C₆alkoxy, C₁-C₆haloalkoxy, C₃-C₆alkenyloxy, C₃-C₆alkynyloxy, C₁-C₆-alkylthio, C₁-C₆haloalkylthio, C₃-C₆alkenylthio, C₃-C₆haloalkenylthio, C₃-C₆alkynylthio, C₁-C₄-alkoxy-C₁-C₂alkylthio, C₁-C₄alkylcarbonyl-C₁-C₂alkylthio, C₁-C₄alkoxycarbonyl-C₁-C₂alkylthio, cyano-C₁-C₄alkylthio, C₁-C₆alkylsulfinyl, C₁-C₆haloalkylsulfinyl, C₁-C₆alkylsulfonyl, C₁-C₆-haloalkylsulfonyl, aminosulfonyl, C₁-C₄alkylaminosulfonyl, di-(C₁-C₄alkyl)aminosulfonyl, R₂₉-C₁-C₃alkylene, NR₃₄R35, halogen, cyano, nitro, phenylthio or benzylthio, wherein phenylthio and benzylthio may themselves be mono-, di- or tri-substituted on the phenyl ring by C₁-C₃alkyl, C₁-C₃haloalkyl, C₁-C₃alkoxy, C₁-C₃haloalkoxy, halogen, cyano or by nitro;
X₂ is oxygen, di-C₁-C₄alkoxy or ═NR₁₂;
R₁₂ is hydroxy or C₁-C₄alkoxy;
R₁₃, R₁₄, R₁₅, R₁₆, R₁₇, R₃₀, R₃₁, R₃₂, R₃₃, R₃₄, R₃₅, R36, R₃₇, R₃₈, R₃₉, R₄₀ and R₄₁ are each independently of the others C₁-C₁₂alkyl;
or R₁₃ and R₁₄ together or R₃₀ and R₃₁ together or R₃₂ and R₃₃ together or R₃₄ and R₃₅ together or R₃₆ and R₃₇ together and/or R₁₆ and R₁₇ together or R₃₉ and R40 together form, with the nitrogen atom to which they are bonded, a 3- to 7-membered ring;
R₁₈ is hydrogen or C₁-C₆alkyl;
R₁₉ is NR₃₇R₃₈, C₁-C₆alkyl, C₁-C₆haloalkyl, C₂-C₆alkenyl, C₂-C₆haloalkenyl, C₃-C₆alkynyl, C₃-C₆haloalkynyl, C₃-C₆cycloalkyl or phenyl, wherein phenyl may itself be substituted by C₁-C₃alkyl, C₁-C₃haloalkyl, C₁-C₃alkoxy, C₁-C₃haloalkoxy, halogen, cyano or by nitro;
R₂₀ and R₂₉ are each independently of the other C₁-C₃alkoxy, C₂-C₄alkoxycarbonyl, C₁-C₃-alkylthio, C₁-C₃alkylsulfinyl, C₁-C₃alkylsulfonyl or phenyl, wherein phenyl may itself be substituted by C₁-C₃alkyl, C₁-C₃haloalkyl, C₁-C₃alkoxy, C₁-C₃haloalkoxy, halogen, cyano or by nitro;
R₂₇ and R₂₈ are each independently of the other hydrogen, C₁-C₆alkyl, C₁-C₆haloalkyl, C₃-C₆alkenyl, C₃-C₆alkynyl, C₁-C₆alkoxy, benzyl or phenyl, wherein benzyl or phenyl may themselves be substituted by C₁-C₄alkyl, C₁-C₄haloalkyl, C₁-C₄alkoxy, C₁-C₄haloalkoxy, C₁-C₄alkylcarbonyl, C₁-C₄alkoxycarbonyl, C₁-C₄alkylthio, C₁-C₄alkylsulfinyl, C₁-C₄alkyl-sulfonyl, C₁-C₄haloalkylthio, C₁-C₄haloalkylsulfinyl, C₁-C₄haloalkylsulfonyl, halogen, nitro or by cyano;
or an agrochemically tolerable salt or any stereoisomer or tautomer of a compound of formula I.

2. A compound of formula I according to claim 1, which corresponds to formula Ib 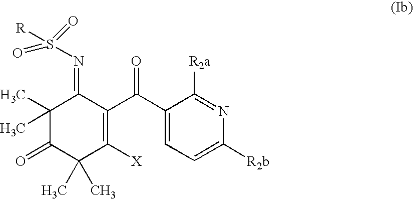
wherein
X is chlorine, C₁-C₈alkylthio, C₃-C₆alkenylthio or phenylthio;
R₂a is C₁-C₃alkyl, fluoromethyl, difluoromethyl, trifluoromethyl, C₁-C₃alkoxymethyl or C₁-C₂-alkoxy-C₁-C₂alkoxymethyl;
R₂b is difluoromethyl, difluorochloromethyl or trifluoromethyl; and
R is C₁-C₆alkyl, C₁-C₄haloalkyl, C₂-C₆alkenyl, phenyl, pyridyl, or phenyl or pyridyl substituted by halogen, C₁-C₄alkyl, C₁-C₄alkoxycarbonyl, C₁-C₄haloalkoxy or by C₁-C₄haloalkyl.

3. Use of a compound of formula Ia 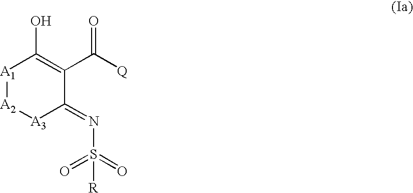
wherein A₁, A₂, A₃, R and Q are as defined for formula I in claim 1, in the preparation of a compound of formula I.

4. A herbicidal and plant-growth-inhibiting composition that comprises a herbicidally effective amount of a compound of formula I on an inert carrier.

5. A method of controlling undesired plant growth, which comprises applying a herbicidally effective amount of a compound of formula I, or of a composition comprising such a compound, to the plants or to the locus thereof.

6. A method of inhibiting plant growth, which comprises applying a herbicidally effective amount of a compound of formula I, or of a composition comprising such a compound, to the plants or to the locus thereof.
